(19) United States
(12) Patent Application Publication
(10) Pub. No.: US 2026/0021142 A1
(43) Pub. Date: Jan. 22, 2026

(54) HYDROGELS HAVING CONTROLLED HYDROLYTIC DEGRADATION

(71) Applicant: GEORGIA TECH RESEARCH CORPORATION, Atlanta, GA (US)

(72) Inventors: Andres J. GARCIA, Atlanta, GA (US); Michael HUNCKLER, Atlanta, GA (US); Karen MARTIN, Oslo (NO)

(21) Appl. No.: 18/995,644

(22) PCT Filed: Jul. 19, 2023
(86) PCT No.: PCT/US2023/070488

§ 371 Date: Jan. 16, 2025

Related U.S. Application Data
(60) Provisional application No. 63/390,503, filed on Jul. 19, 2022.

(51) Int. Cl. A61K 35/28; A61K 9/06; A61K 47/10; A61K 47/65

(52) U.S. Cl. CPC A61K 35/28; A61K 9/06; A61K 47/10; A61K 47/65

(57) ABSTRACT

A hydrogel is provided comprising a first polymer, a second polymer, and a crosslinker. The first polymer comprises at least one ester-containing moiety and at least one first crosslinking moiety. The second polymer comprises at least one amide-containing moiety and at least one second crosslinking moiety. The crosslinker is covalently bound to the first polymer and the second polymer via the first crosslinking moiety and the second crosslinking moiety. Articles formed from the hydrogel as well as methods of use are also described.

### CROSS-REFERENCE TO RELATED APPLICATIONS

This application is a U.S. National Stage application filed under 35 U.S.C. § 371 of PCT/US2023/070488, filed Jul. 19, 2023, which claims the benefit of priority to U.S. Provisional Application No. 63/390,503, filed Jul. 19, 2022, the disclosures of which are incorporated herein by reference in their entirety.

### STATEMENT REGARDING FEDERALLY SPONSORED RESEARCH OR DEVELOPMENT

This invention was made with government support under Grant No. R01DK128840 awarded by the National Institutes of Health. The Government has certain rights in the invention.

### TECHNICAL FIELD

This disclosure relates to compositions and, more particularly, to hydrogels having tunable degradation.

### BACKGROUND

Hydrogels find use in many medical, therapeutic, and biological applications. Typically, there is a desire for hydrogels used in such applications to degrade over time, particularly in medical applications where permanent implantation is undesired. However, available hydrogel technologies often have unpredictable degradation kinetics that make them difficult to use in such applications. Thus, there is a clear need for the development of biologically compatible hydrogels whose degradation may be specifically tuned for any particular application in which they are applied.

### SUMMARY

In accordance with the purposes of the disclosed materials, compounds, compositions, and methods, as embodied and broadly described herein, the disclosed subject matter, in one aspect, relates to compositions, articles formed from said compositions, methods of making said compositions, and methods of using said compositions. In particular, the present disclosure is related to hydrogels and methods of using the same.

In one aspect, a hydrogel is provided comprising a first polymer, a second polymer, and a crosslinker. In some aspects, the first polymer comprises at least one ester-containing moiety and at least one first crosslinking moiety. In some aspects, the second polymer comprises at least one amide-containing moiety and at least one second crosslinking moiety. In some aspects, the crosslinker is covalently bound to the first polymer and the second polymer via the first crosslinking moiety and the second crosslinking moiety.

In another aspect, a therapeutic delivery composition is provided comprising a hydrogel described herein and one or more therapeutic agents.

In another aspect, cell culture mediums, tissue scaffolds, or wound dressings are provided comprising a hydrogel described herein.

In another aspect, a method of delivering one or more therapeutic agents to a target site in a subject is provided, the method comprising administering a therapeutically effective amount of a therapeutic delivery composition described herein.

In another aspect, a method is provided for promoting tissue growth in a target site in a subject in need thereof, comprising administering to the target site a therapeutically effective amount of a hydrogel described herein.

In another aspect, a method is provided of transplanting at least one cell to a target site in a subject in need thereof, the method comprising administering to the target site an effective amount of a composition comprising the hydrogel described herein and the at least one cell.

The details of one or more embodiments of the disclosure are set forth in the accompanying drawings and the description below. Other features, objects, and advantages of the disclosure will be apparent from the description, drawings, and claims.

### DESCRIPTION OF DRAWINGS

FIGS. 1A-1D depict the fabrication and characterization of exemplary hydrogels disclosed herein. (FIG. 1A) PEG-4MAL macromers, terminated with amide-maleimide (aMAL) or ester-maleimide (eMAL), are mixed and reacted with free thiol-containing adhesive peptides or growth factors, and crosslinked with nondegradable DTT. The thio-maleimide click reaction drives the synthesis of these hydrogels. (FIG. 1B) FTIR spectra of lyophilized hydrogels (without adhesive peptides) reveal ester and amide peaks. (FIG. 1C) Equivalent mechanical properties of PEG-4eMAL and PEG-4aMAL gels with varying polymer density (p=0.32, two-way ANOVA). (FIG. 1D) Varying the ratio of eMAL/aMAL yields equivalent mechanical properties in 5% (w/v) PEG-4MAL hydrogels (p—0.21, one-way ANOVA). All data is presented as mean±s.d., with n=10 gels/group.

FIGS. 2A-2E depict the in vivo degradation kinetics of representative PEG-4MAL hydrogels. (FIG. 2A) Schematic of hydrogel fabrication with PEG-4MAL (aMAL and eMAL), DTT crosslinker, and a near-infrared fluorophore. The components were mixed prior to injection into the dorsal subcutaneous pocket of mice. (FIG. 2B) A subset of hydrogels (n=5/group; aMAL and eMAL) were harvested from the subcutaneous pocket 30 min after injection, swollen, and rheologically measured. Data presented as mean±s.d. and analyzed with unpaired t-test (p=0.56). (FIG. 1C) PEG-4aMAL and PEG-4eMAL were combined in various ratios (0, 25, 50, 75, 100% eMAL) prior to subcutaneous injection (n=5-6 gels/group) and fluorescence monitored for 35 days. Representative fluorescent images or photos of mouse dorsum following hydrogel injection. (FIG. 2D) Normalized radiant efficiency of fluorescent hydrogels and exponential decay curve fit demonstrate tunability of degradation kinetics. Data presented as mean (points)±s.d. (shaded area). (FIG. 2E) Half-life of hydrogels calculated from the normalized radiant efficiency. Data presented as mean±s.d. and analyzed with one-way ANOVA followed by Tukey's multiple comparison analysis, *p<0.001.

FIGS. 3A-3F depict the fabrication and in vivo and ex vivo characterization of representative proteolytic hydrogels. (FIG. 3A) PEG-4MAL macromers, terminated with amide-maleimide (aMAL), are mixed and reacted with free thiol-containing adhesive peptides (RGD), and crosslinked with a proteolytically degradable crosslinker, VPM. The thiol-maleimide click reaction drives the synthesis of these hydrogels. (FIG. 3B) Schematic of the in vivo evaluation of VPM-crosslinked PEG-4aMAL hydrogels. Hydrogel components were mixed and injected onto the fat pad or into the subcutaneous pocket. (FIGS. 3C and 3D) The VPM-crosslinked hydrogels (75 μL hydrogels comprised of 5% w/v PEG+1mMRGD) remained present and marginally degraded at 6-7 weeks post-transplantation (arrows denote nondegraded hydrogel). (FIG. 3E) At 4 weeks, a VPM-crosslinked PEG-4aMAL hydrogel transplanted subcutaneously has developed a thick fibrotic capsule surrounding the hydrogel. (FIG. 3F) PEG-4MAL hydrogels (50 μL, 5% w/v) were crosslinked with VPM (protease-degradable crosslinker) or DTT (nondegradable crosslinker) in the subcutaneous space. At week 8, the hydrogels were explanted, thoroughly rinsed for 24 hrs in diH₂O, and lyophilized. VPM-crosslinked hydrogels exhibited reduced mass, indicating some degradation, but still retained considerable structure in the subcutaneous space (p<0.001, t-test).

FIGS. 4A-4C depict in vivo degradation kinetics of PEG-4MAL (50% aMAL+50% eMAL) hydrogels with a range of PEG polymer density. (FIG. 4A) Schematic of hydrogel fabrication with PEG-4MAL (aMAL and eMAL), DTT crosslinker, and a near-infrared fluorophore. The components were mixed prior to injection into the dorsal subcutaneous pocket of mice. (FIG. 4B) PEG-4MAL (50% aMAL+50% eMAL) hydrogels were fabricated at a range of PEG polymer density (4%, 5%, 6%, 7%, 8% w/v) immediately prior to subcutaneous injection (n=5-6 gels/group) and fluorescence monitored for 35 days. Normalized radiant efficiency of fluorescent hydrogels and exponential decay curve fit demonstrate some tunability of degradation kinetics with initial polymer density. Data presented as mean (points)±s.d. (shaded area). (FIG. 4C) Half-life of hydrogels calculated from the normalized radiant efficiency. Data presented as mean±s.d. and analyzed with one-way ANOVA followed by Tukey's multiple comparison analysis, *p<0.05, ** p<0.01.

FIGS. 5A-5E depict in vitro degradation of PEG-4MAL hydrogels with a variety of mediums. (FIG. 5A) Schematic of hydrogel degradation experiment in PBS. 5% PEG-4MAL hydrogels (75 μL) with 100% aMAL, 50/50% aMAL/eMAL, or 100% eMAL were crosslinked with DTT and swollen in PBS−/− (pH 7) at room temperature. (FIG. 4B) At various time points, the hydrogels were removed from PBS, rinsed (dH₂O for 6 hrs), and lyophilized. Hydrogels did not exhibit degradation up to day 28 (n=5 gels per group per timepoint; two-way ANOVA, p=0.3). (FIG. 5C) Schematic of hydrogel degradation experiment in serum: heat-inactivated fresh mouse serum (HI-MS), fresh mouse serum (MS), and commercial heat-inactivated fetal bovine serum (HI-FBS). 5% PEG-4MAL hydrogels (100 μL) were crosslinked with DTT and swollen in PBS prior to incubation in serum at 37° C. for 24 hrs. (FIGS. 5D and 5E) After 24 hrs, eMAL hydrogels completely degraded in fresh non-heat-inactivated-mouse-serum (MS), while all other conditions were equivalent (scalebar=1 cm).

FIG. 6 depicts a PEG-4MAL hydrogel synthesis scheme. Hydrogels are prepared by reacting a 4-arm 20 kDa PEG macromer with terminal maleimide groups (PEG-4MAL) with cysteine-containing cell adhesive peptide RGD and the proteolytically degradable crosslinker VPM, cysteine-terminated on each end, via a Michael-type addition reaction. The terminal maleimides are linked to the PEG backbone via either amide (PEG-4aMAL) or hydrolytically-degradable ester groups (PEG-4eMAL).

FIGS. 7A-7I depicts how PEG-4MAL hydrogels synthesized with hydrolytic ester linkers overcome degradation challenges of amide-linked hydrogels and enable cutaneous wound repair. (FIG. 7A) Schematic of subcutaneous hydrogel degradation study. PEG-4MAL hydrogels were synthesized with different ratios of ester- and amide-linked macromers, labeled in the figure as percent PEG-4eMAL. Maleimide-terminated dye was tethered into the gel to enable in vivo tracking by IVIS. Hydrogel components were injected subcutaneously and polymerized in situ in wild-type C57BL/6J mice on day 0 and tracked for 28 days. (FIG. 7B) IVIS images of fluorescently tagged PEG-4a/eMAL hydrogels over 7 days following subcutaneous injection. Hydrogel locations: top left=100% PEG-4eMAL, top right=75% PEG-4eMAL, bottom left=50% PEG-4eMAL, and bottom right=100% PEG-4aMAL. (FIG. 7C) Quantification of fluorescence within a region of interest (ROI) of PEG-4a/eMAL hydrogels normalized to the day 0 radiant efficiency of the ROI (n=4 hydrogels per group). Red arrow indicates day of hydrogel injection. Plotted as mean for each time point±SD in the shaded region. A repeated measures ANOVA was used to detect differences followed by Tukey's multiple comparisons test with adjustment for multiple comparisons. * p<0.05, ** p<0.01, *** p<0.001. (FIG. 7D) Hydrogel degradation over time plotted for each individual gel. (FIG. 7E) In vivo degradation half-life of PEG-4a/eMAL hydrogels calculated from the fluorescence signal curves (one phase decay, least squares fit). A one-way ANOVA was used to detect differences followed by Tukey's multiple comparisons test. (FIG. 7F) Schematic of full-thickness skin wound experiment. 4 mm full-thickness skin wounds were made on the upper dorsal region of wild-type C57BL/6J mice. 30 μL of PEG-4MAL hydrogel was introduced into the wound immediately after surgery and 3 days post-operatively. Wound healing was tracked for up to 11 days. (FIG. 7G) Wound images. (FIG. 7H) Wound closure of full-thickness skin wounds as a percent of day 0 wound size (n=7 wounds per group). Red arrows indicate days of hydrogel was added to the wound. Plotted as mean for each time point±SD in the shaded region. (FIG. 7I) Wound healing over time plotted for each individual wound. A repeated measures ANOVA was used to detect differences followed by Tukey's multiple comparisons test. * p<0.05, *** p<0.001, **** p<0.0001.

FIGS. 8A-8E depict how the hydrogel degradation rate in diabetic mice can be tuned by altering the ratio of ester-linked to amide-linked macromer. (FIG. 8A) Schematic of subcutaneous hydrogel degradation study. PEG-4MAL hydrogels were synthesized with different ratios of ester- and amide-linked macromers, labeled in the figure as percent PEG-4eMAL. Maleimide-terminated AlexaFluor 750 was tethered into the gel to enable in vivo tracking by IVIS. Hydrogel components were injected subcutaneously and polymerized in situ in obese, diabetic db/db mice on day 0, and degradation was tracked for 15 days. (FIG. 8B) IVIS images of fluorescently tagged PEG-4a/eMAL hydrogels over 15 days following subcutaneous injection. (FIG. 8C) Quantification of fluorescence within a region of interest (ROI) of PEG-4a/eMAL hydrogels normalized to the day 0 radiant efficiency of the ROI (n=3). Red arrow indicates day of hydrogel injection. Plotted as mean for each time point±SD in the shaded region. A mixed-effects model (REML) was used to detect differences followed by Tukey's multiple comparisons test with adjustment for multiple comparisons. *p<0.05, ** p<0.01. P-values are given for day 15. (FIG. 8D) Hydrogel degradation over time plotted for each individual gel. (FIG. 8E) In vivo degradation half-life of PEG-4a/eMAL hydrogels calculated from the fluorescent signal curves (one phase decay, least squares fit). A one-way ANOVA was used to detect statistical differences between the 50%, 75%, and 100% groups followed by Tukey's multiple comparisons test with adjustment for multiple comparisons.

FIGS. 9A-9D depict how the hydrolytic hydrogels outperform proteolytically-degradable hydrogels for in vivo murine mesenchymal stromal cells (mMSC) delivery. (FIG. 9A) Schematic of mMSC persistence studies. 50 μL hydrogels containing 1.5×10⁵ mMSC^{fluc} were injected into the dorsal subcutaneous space of obese, diabetic db/db mice. Persistence of mMSC^{fluc} was tracked by IVIS bioluminescence imaging. (FIG. 9B) Bioluminescence images of mMSC^{fluc} after subcutaneous injection in db/db mice. (FIG. 9C) Normalized bioluminescence of transplanted mMSC^{fluc} over time upon subcutaneous injection in db/db mice. Red arrow indicates day of hydrogel injection. Plotted as mean for each time point±SD in the shaded region (n=5 samples per condition). (FIG. 9D) mMSC^{fluc} persistence over time plotted for each individual sample. A repeated measures ANOVA was used to detect differences followed by Tukey's multiple comparisons test. ** p<0.01, **** p<0.0001.

FIGS. 10A-10G depict how delivery of mMSC within hydrolytic PEG-4MAL hydrogels improves cutaneous wound healing in diabetic mice. (FIG. 10A) Schematic of full-thickness skin wound experiment. 4 mm full-thickness skin wounds were made on the upper dorsal region of obese, diabetic db/db mice. 30 μL of PEG-4MAL hydrogel containing mMSC or cell-free control was introduced into the wound immediately after surgery. Wound healing was tracked for up to 11 days. (FIG. 10B) Wound images. (FIG. 10C) Closure of full-thickness skin wounds as a percent of day 0 wound size following delivery of 75% ester-linked 25% amide-linked PEG hydrogels containing either no cells, 2.5×10⁴, or 2.5×10⁵ mMSC (n=6-7 wounds per condition). Red arrow indicates day of hydrogel injection. Plotted as mean for each time point±SD in the shaded region. A two-way ANOVA was used to detect differences followed by Tukey's multiple comparisons test. *p<0.05 at day 11. (FIG. 10D) Normalized wound closure by treatment group. Each line represents an individual wound. (FIG. 10E) Quantification of wound size as a percent of day 0 on days 9 and 11 (n=6-7 wounds per condition); mean±SD. (FIG. 10F) H&E-stained histology images of full-thickness skin wounds treated with hydrogel-delivered mMSC. (FIG. 10G) Quantification of epidermal thickness in histology images. Points indicate measurements (n=5) for each mouse (bar, n=3 per group). A nested ANOVA was used to detect differences followed by Tukey's multiple comparisons test. * p<0.05, **** p<0.0001.

FIGS. 11A-11F depict how hydrogel-delivery of mMSC alters mononuclear phagocyte recruitment and phenotype in diabetic full-thickness skin wounds. (FIG. 11A) Schematic of full-thickness skin wound experimental set-up. 4 mm full-thickness skin wounds were made on the upper dorsal region of obese, diabetic db/db mice. 30 μL of saline or 75% ester-linked 25% amide-linked PEG-4MAL hydrogel with or without 2.5×10⁵ mMSC was introduced into the wound immediately after surgery. On day 3 post-surgery, immune populations infiltrating the wound were assessed by spectral flow cytometry. Wound-infiltrating CD45⁺CD11b⁺Siglec-F⁻Ly6G⁻ mononuclear macrophages were identified, and the FlowSOM algorithm was used to cluster mononuclear phagocytes into sub-populations. (FIG. 11B) tSNE plot showing mononuclear phagocyte populations colored by FlowSOM-identified clusters. Clusters were identified as Mono (monocyte, Ly6C^{hi}F4/80 CX3CR1^{lo}) or Mac (macrophage, Ly6C⁻F4/80⁺CX3CR1⁺). (FIG. 11C) tSNE maps colored by expression of markers used to determine mononuclear phagocyte FlowSOM clusters. (FIG. 11D) Number of infiltrating cells in each FlowSOM-identified cluster (n=5-6 wounds per condition); mean±SD. A one-way ANOVA was used to detect differences followed by Tukey's multiple comparisons test. * p<0.05. (FIG. 11E) Expression of mononuclear phagocyte phenotype markers in the FlowSOM-identified clusters. Color indicates median fluorescence intensity, size indicates p-value between samples in the saline alone and 75% PEG-4eMAL+mMSC groups. A two-way ANOVA was used to detect differences followed by Tukey's multiple comparisons test. (FIG. 11F) Median fluorescence intensity of CD9 and CD86 by FlowSOM-identified cluster and experimental group. A two-way ANOVA was used to detect differences followed by Tukey's multiple comparisons test. * p<0.05, ** p<0.01, *** p<0.001, **** p<0.0001.

FIG. 12A-13C depicts the degradation of 100% PEG-4eMAL gels crosslinked with either VPM or DTT. (FIG. 12A) Quantification of fluorescence within a region of interest (ROI) of PEG-4a/eMAL hydrogels normalized to the day 0 radiant efficiency of the ROI (n=3). Plotted as mean for each time point±SD in the shaded region. A repeated measures ANOVA was used to detect differences followed by Tukey's multiple comparisons test. *** p<0.001, **** p<0.00*1. (FIG. 12B) Hydrogel degradation over time plotted for each individual gel. (FIG. 12C) In vivo degradation half-life of PEG-4eMAL hydrogels calculated from the fluorescence signal curves (one phase decay, least squares fit). A student's t-test was used to detect statistical differences.

FIG. 13 depicts the mechanical properties of different hydrogel formulations polymerized in vitro. A one-way ANOVA was used to detect differences followed by Tukey's multiple comparisons test. No statistical differences were found between any of the groups (n=14-17 gels per condition; p=0.0779).

FIG. 14 depicts the mass and swelling ratio of explanted PEG-4aMAL. 50 μL of PEG-4aMAL (0% PEG-4eMAL) hydrogel solution was injected into the mouse dorsum and allowed to polymerize in situ. Hydrogels were explanted following polymerization on day 0 or after 28 days in vivo. A student's t-test was used to detect differences (n=3-4 gels per condition).

FIGS. 15A-15E depict how ester-linked hydrogels achieve equivalent wound repair times to saline in db/db mice. (FIG. 15A) Schematic of full-thickness skin wound experiment. 4 mm full-thickness skin wounds were made on the upper dorsal region of obese, diabetic db/db mice and heterozygous littermate control db/m+ mice. PEG-4MAL hydrogel (30 μL) was polymerized into the wound immediately after surgery and 3 days post-operatively. Wound healing was tracked for up to 14 days. (FIG. 15B) Cutaneous wound images. Scale bar equals 5 cm. (FIG. 15C) Closure of full-thickness skin wounds as a percent of day 0 wound size. (FIG. 15D) Wound healing over time plotted for each individual wound. (FIG. 15E) Quantification of wound size on day 9 (n=4-5); mean±SD. A one-way ANOVA was used to detect differences followed by Tukey's multiple comparisons test. * p<0.05, ** p<0.01 vs. db/m+ saline; #p<0.05, ##p<0.01 vs. db/m+50% PEG-4eMAL.

FIGS. 16A-16B depict the murine mesenchymal stem/stromal cell characterization. (FIG. 16A) Histograms for murine MSC markers evaluated by flow cytometry with surface marker-specific and isotype control antibodies. (FIG. 16B) Microscopy images of mMSC stained with Alizarin Red following culture in osteogenic media for 9 days.

FIGS. 17A-17D depict how hydrolytic hydrogels enhance mMSC persistence at site of injection compared to bolus injections. (FIG. 17A) Schematic of mMSC persistence studies. 50 μL of hydrogel solution or saline containing 1.5×105 mMSCfluc were injected into the dorsal subcutaneous space of wild-type C57BL/6J mice. Persistence of mMSCfluc was tracked by IVIS bioluminescent imaging. (FIG. 17B) IVIS images of mMSCfluc after subcutaneous injection in wild-type C57BL/6J mice. (FIG. 17C) Normalized bioluminescence of transplanted mMSCfluc over time upon subcutaneous injection in wild-type C57BL/6J mice. Plotted as mean for each time point±SD in the shaded region (n=4 samples per condition). (FIG. 17D) mMSCfluc persistence over time plotted for each individual sample. A repeated measures ANOVA was used to detect differences followed by Tukey's multiple comparisons test. *** p<0.001.

FIGS. 18A-18F depict how hydrolytic gel delivery of mMSC alters the recruitment of myeloid cells to diabetic full-thickness skin wounds. (FIG. 18A) Flow cytometry gating strategy used to identify the CD45+CD11b+ myeloid population. This population was exported as an FCS file and imported into R for use with the CATALYST package. (FIG. 18B) tSNE plot showing myeloid populations colored by FlowSOM-identified clusters. Clusters were identified as Eosinophil (Siglec-F+), Neutrophil (Ly6G+Ly6C+), F4/80+ (macrophages, Siglec-F−Ly6G-Ly6Clo), Ly6C+ (monocytes, Siglec-F−Ly6G-F4/80lo), F4/80+Ly6Cmid (monocytes, Siglec-F−Ly6G−), and other. (FIG. 18C) tSNE maps colored by expression of markers used to determine myeloid FlowSOM clusters. (FIG. 18D) Number of infiltrating cells in each FlowSOM-identified cluster (n=5-6 wounds per condition); mean±SD. A one-way ANOVA was used to detect statistical differences followed by Tukey's multiple comparisons test with adjustment for multiple comparisons. *p<0.05, ** p<0.01. (FIG. 18E) Heatmap showing median scaled expression of all markers in each FlowSOM-identified cluster. FlowSOM-identified clusters and markers are grouped using hierarchical clustering. (FIG. 18F) Histograms showing expression of key myeloid markers in each of the FlowSOM-identified clusters.

FIGS. 19A-19C depict marker expression in FlowSOM-identified mononuclear phagocyte clusters. (FIG. 19A) Heatmap showing median scaled expression of all markers in each FlowSOM-identified cluster. FlowSOM-identified clusters and markers are grouped using hierarchical clustering. (FIG. 19B) Histograms showing expression of key mononuclear phagocyte markers in each of the FlowSOM-identified clusters. (FIG. 19C) Histograms showing expression of mononuclear phagocyte phenotype markers in each of the FlowSOM-identified clusters.

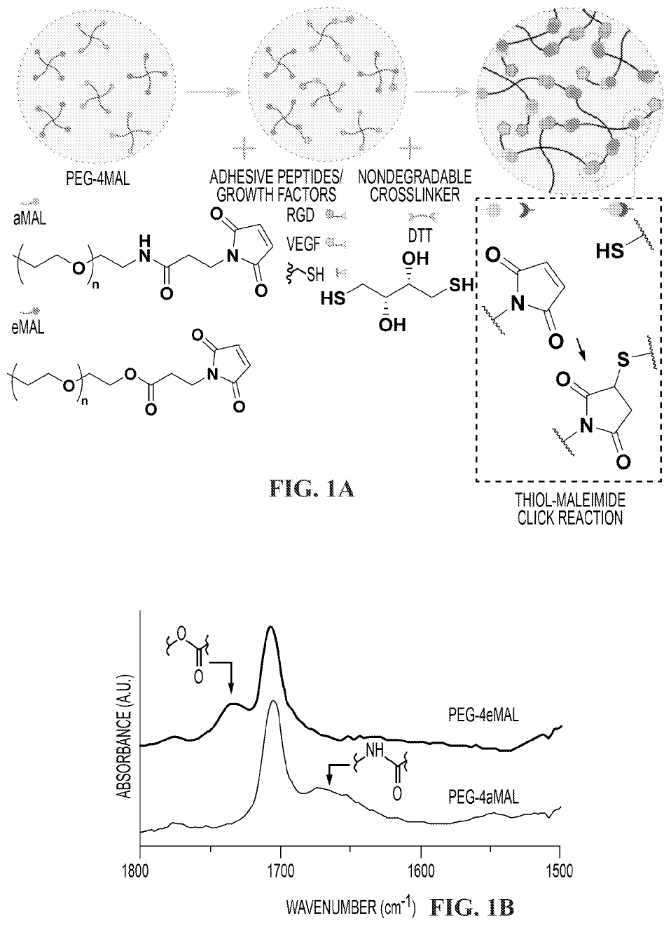

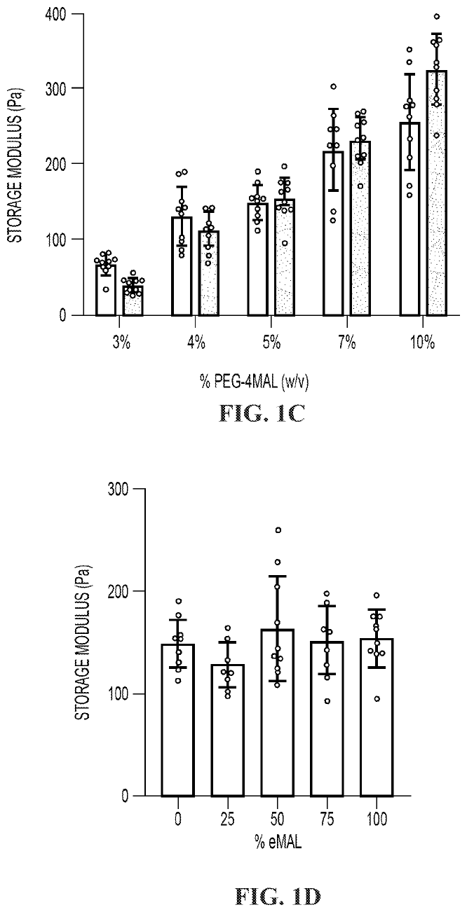

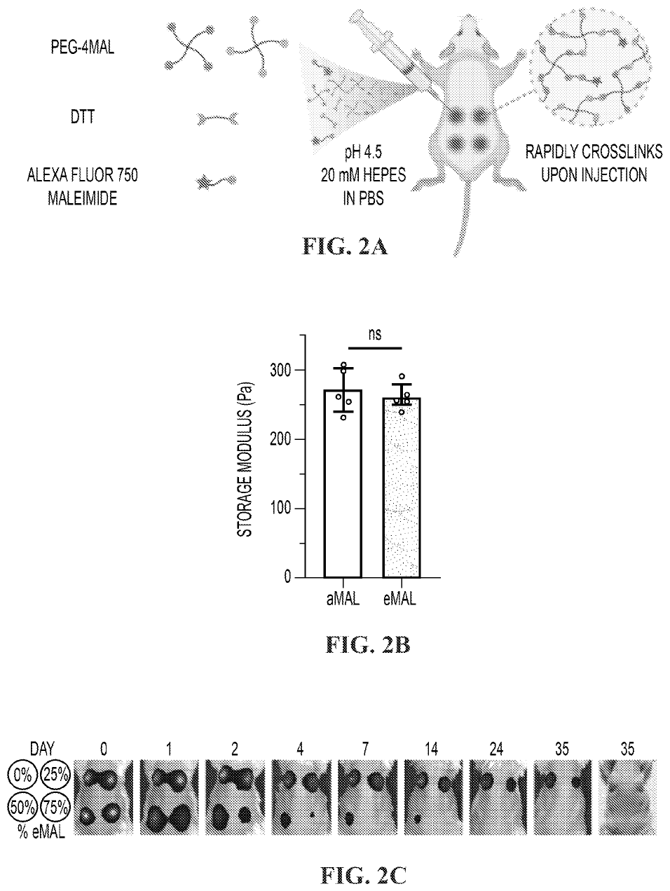

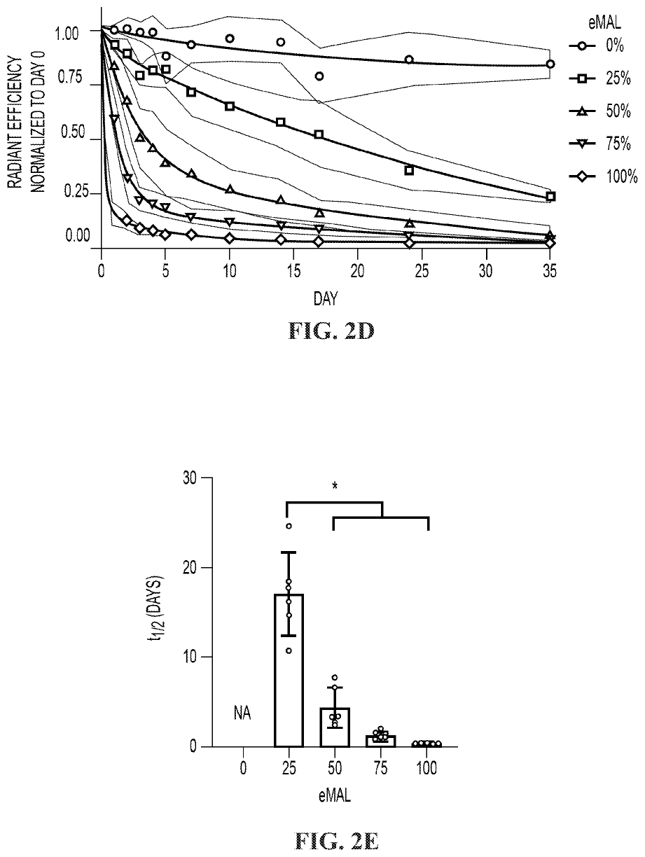

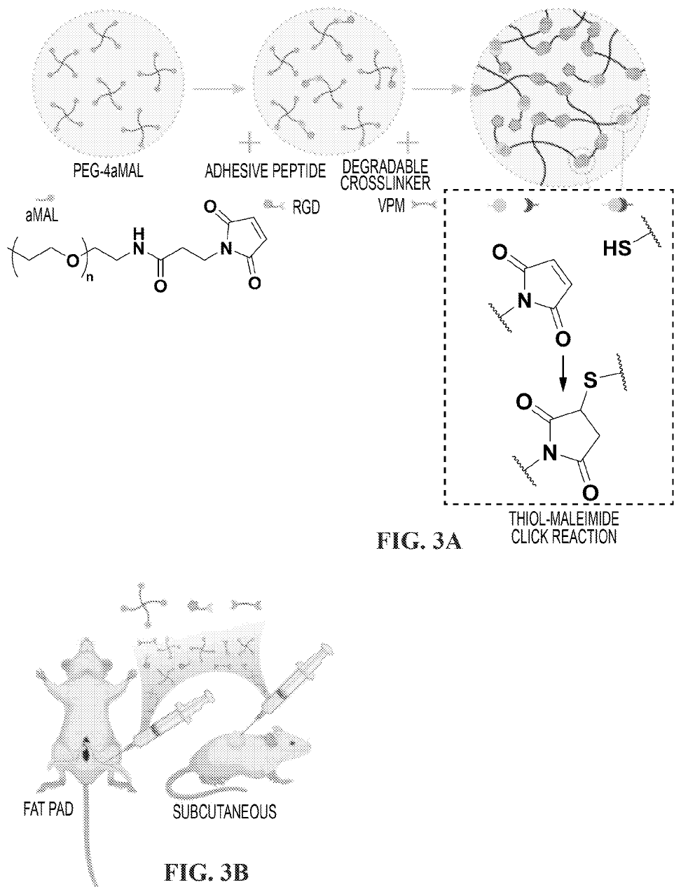

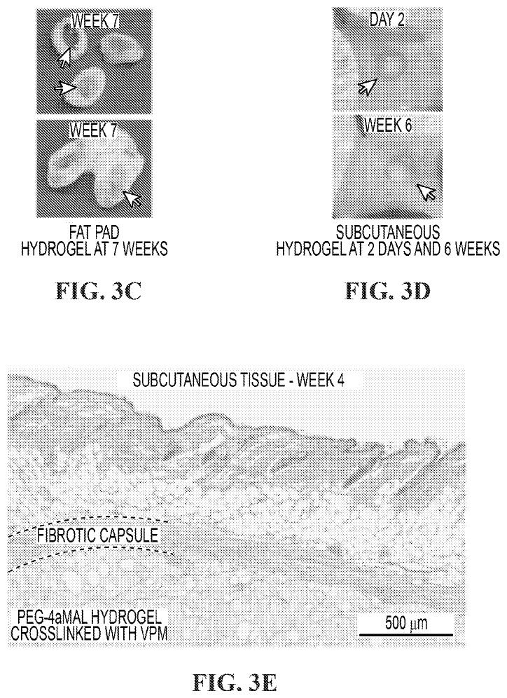

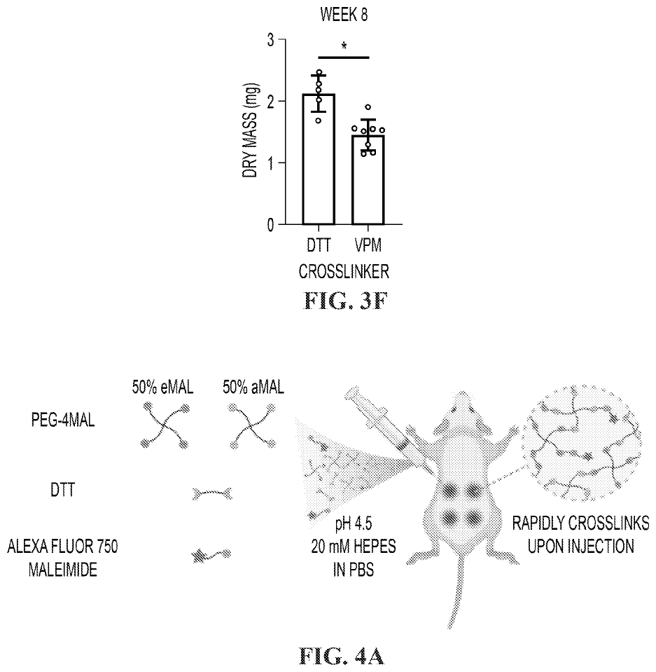

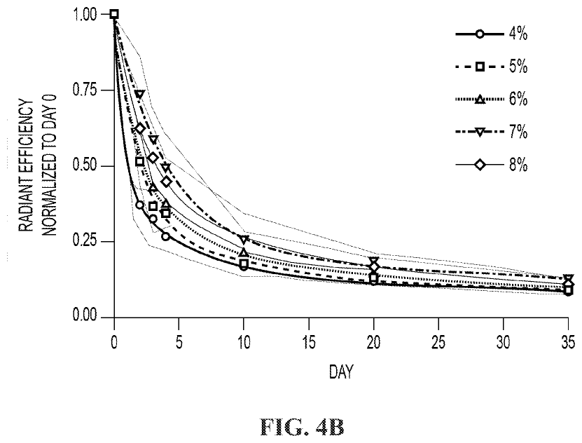

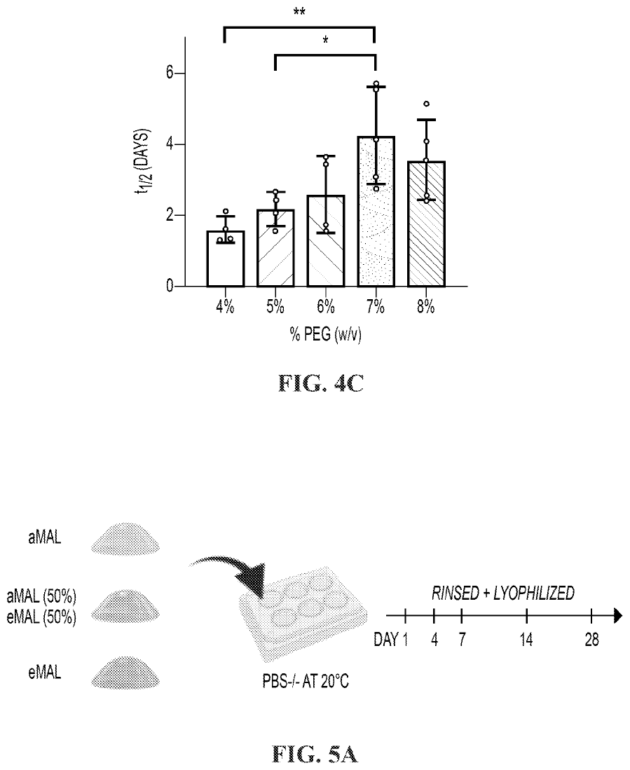

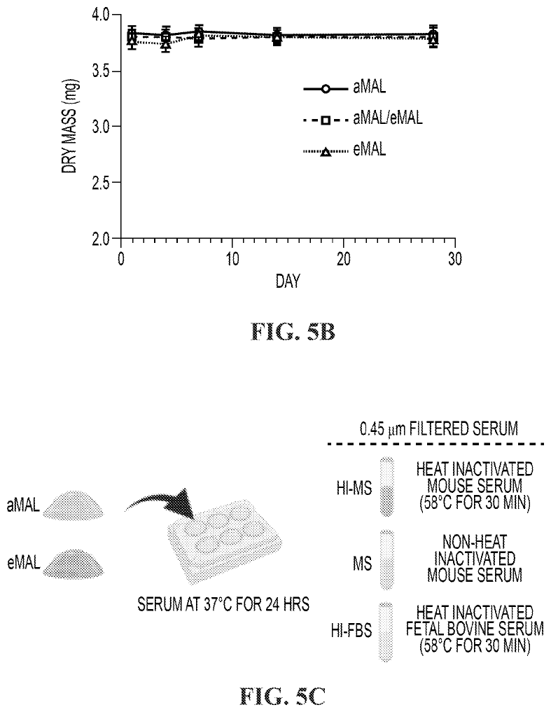

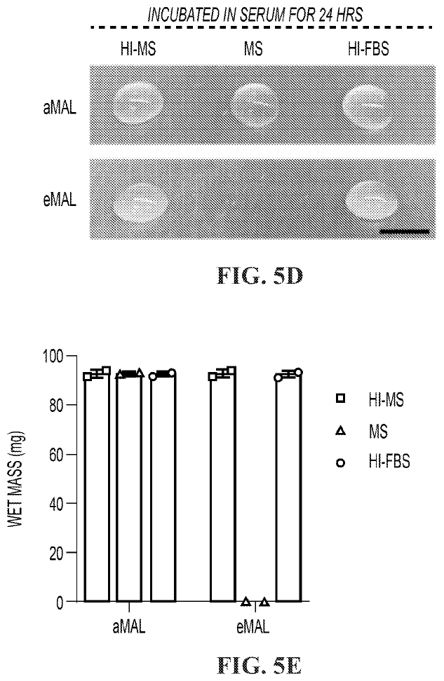

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain, benefiting from the teachings presented in the descriptions herein and the associated drawings. Therefore, it is understood that the disclosures are not limited to the specific embodiments disclosed and that modifications and other embodiments are intended to be included within the scope of the appended claims.

The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual embodiments described and illustrated herein has discrete components and features that may be readily separated from or combined with the features of any of the other several embodiments without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Nothing herein is to be construed as an admission that the present invention is not entitled to antedate such publication by prior invention. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of.”

As used in the specification and the appended claims, the singular forms “a,” “an,” and “the” include plural referents unless the context dictates otherwise.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’ ‘about y,’ and ‘about z’ as well as the ranges of ‘less than x,’ ‘less than y.’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,′ and ‘greater than z.’ In addition, the phrase “about ‘x’ to ‘y’,” where ‘x’ and ‘y’ are numerical values, includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, the term “therapeutically effective amount” refers to an amount sufficient to achieve the desired therapeutic result or to have an effect on undesired symptoms but generally insufficient to cause adverse side effects. The specific therapeutically effective dose level for any particular patient will depend upon a variety of factors, including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the particular compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to permanently halt the progression of the disease. This can be monitored by routine diagnostic methods known to one of ordinary skill in the art for any particular disease. The desired response to treatment of the disease or condition can also be delaying the onset or even preventing the onset.

For example, it is well within the skill of the art to start doses of a compound at levels lower than those required to achieve the desired therapeutic effect and to increase the dosage gradually until the desired effect is achieved. If desired, the effective daily dose can be divided into multiple doses for administration. Consequently, single-dose compositions can contain such amounts or submultiples thereof to make up the daily dose. The individual physician can adjust the dosage in the event of any contraindications. It is generally preferred that a maximum dose of the pharmacological agents of the invention (alone or in combination with other therapeutic agents) be used, that is, the highest safe dose according to sound medical judgment. However, a patient may insist on a lower or tolerable dose for medical reasons, psychological reasons, or virtually any other reason.

A response to a therapeutically effective dose of a disclosed compound or composition can be measured by determining the physiological effects of the treatment or medication, such as the decrease or lack of disease symptoms following the administration of the treatment or pharmacological agent. Other assays will be known to one of ordinary skill in the art and can be employed for measuring the level of the response. The amount of a treatment may be varied, for example, by increasing or decreasing the amount of a disclosed compound or pharmaceutical composition, changing the disclosed compound or pharmaceutical composition administered, changing the route of administration, changing the dosage timing, and so on. Dosage can vary and can be administered in one or more dose administrations daily for one or several days. Guidance can be found in the literature for appropriate dosages for given classes of pharmaceutical products.

As used herein, “optional” or “optionally” means that the subsequently described event or circumstance can or cannot occur. The description includes instances where said event or circumstance occurs and those where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to a human and constituents thereof.

As used herein, “treating” and “treatment” generally refer to obtaining a desired pharmacological or physiological effect. The effect can be but does not necessarily have to be prophylactic in preventing or partially preventing a disease, symptom, or condition. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and prophylactic treatment. Those in need of treatment (i.e., subjects in need thereof) can include those already with the disorder or those in which the disorder is to be prevented. As used herein, the term “treating” can include inhibiting the disease, disorder, or condition, e.g., impeding its progress, and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound or a pharmaceutical composition thereof calculated to produce the desired response or responses in association with its administration.

As used herein, “therapeutic” can refer to treating, healing, or ameliorating a disease, disorder, condition, or side effect or decreasing the rate of advancement of a disease, disorder, condition, or side effect.

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates, and other isomers, such as rotamers, as if each is specifically described unless otherwise indicated or otherwise excluded by context. It is to be understood that the compounds provided herein may contain chiral centers. Such chiral centers may be of either the (R-) or (S-) configuration. The compounds provided herein may either be enantiomerically pure or diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to the administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted,” as used herein, means that any one or more hydrogens on the designated atom or group are replaced with a moiety selected from the indicated group, provided that the designated atom's normal valence is not exceeded and the resulting compound is stable. For example, when the substituent is oxo (i.e., ═O), then two hydrogens on the atom are replaced. For example, a pyridyl group substituted by oxo is a pyridine.

Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl) C₀-C₄alkyl, or —C₀-C₄ (C₃-C₇cycloalkyl), the indicated group, in this case cycloalkyl, is either directly bound by a single covalent bond (Coalkyl), or attached by an alkyl chain, in this case 1, 2, 3, or 4 carbon atoms. Alkyls can also be attached via other groups such as heteroatoms, as in —O—C₀-C₄alkyl (C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In some embodiments, the alkyl group is optionally substituted as described herein. The term “alkyl” as used herein is not intended to be limited to monovalent radicals and may include polyvalent radical groups as appropriate, such as divalent, trivalent, tetravalent, pentavalent, and hexavalent alkyl, and the like, based on the position and location of such groups in the compounds described herein as would be readily understood by the skilled person.

“Cycloalkyl” is a saturated or partially unsaturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In some embodiments, the cycloalkyl group is optionally substituted as described herein. The term “cycloalkyl” as used herein is not intended to be limited to monovalent radicals and may include polyvalent radical groups as appropriate, such as divalent, trivalent, tetravalent, pentavalent, and hexavalent cycloalkyl, and the like, based on the position and location of such groups in the compounds described herein as would be readily understood by the skilled person.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein. The term “alkenyl” as used herein is not intended to be limited to monovalent radicals and may include polyvalent radical groups as appropriate, such as divalent, trivalent, tetravalent, pentavalent, and hexavalent alkenyl, and the like, based on the position and location of such groups in the compounds described herein as would be readily understood by the skilled person.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein. The term “alkynyl” as used herein is not intended to be limited to monovalent radicals and may include polyvalent radical groups as appropriate, such as divalent, trivalent, tetravalent, pentavalent, and hexavalent alkynyl, and the like, based on the position and location of such groups in the compounds described herein as would be readily understood by the skilled person.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—).

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein. The term “aryl” as used herein is not intended to be limited to monovalent radicals and may include polyvalent radical groups as appropriate, such as divalent, trivalent, tetravalent, pentavalent, and hexavalent aryl, and the like, based on the position and location of such groups in the compounds described herein as would be readily understood by the skilled person.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S-, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms. The term “heterocycle” as used herein is not intended to be limited to monovalent radicals and may include polyvalent radical groups as appropriate, such as divalent, trivalent, tetravalent, pentavalent, and hexavalent heterocycle, and the like, based on the position and location of such groups in the compounds described herein as would be readily understood by the skilled person.

“Heteroaryl” refers to a stable monocyclic, bicyclic, or multicyclic aromatic ring which contains from 1 to 4, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some embodiments from 1 to 3 or from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one embodiment, the total number of S and O atoms in the heteroaryl ring is not more than 2. In another embodiment, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The term “heteroaryl” as used herein is not intended to be limited to monovalent radicals and may include polyvalent radical groups as appropriate, such as divalent, trivalent, tetravalent, pentavalent, and hexavalent heteroaryl, and the like, based on the position and location of such groups in the compounds described herein as would be readily understood by the skilled person.

The present disclosure provides hydrogels with tunable degradation properties. By varying the ratio of the constituent polymer components of the hydrogel, the degradation rate of the hydrogel may be altered to suit desired applications. Further provided are methods of making and using such polymers.

In one aspect, a hydrogel is provided comprising:
a first polymer;
a second polymer; and
and a crosslinker covalently bound to the first polymer and the second polymer.

As is known in the art, a hydrogel is a polymer network formed by crosslinking one or more multifunctional molecules or polymers. The resulting polymeric network is hydrophilic and swells in an aqueous environment, thus forming a gel-like material, i.e., hydrogel. Hydrogels are characterized by their water insolubility, hydrophilicity, high water absorbability, and swellable properties. The molecule components, units, or segments of a hydrogel are characterized by a significant portion of hydrophilic components, units, or segments, such as segments capable of hydrogen bonding or having ionic species or dissociable species, such as acids (e.g., carboxylic acids, phosphonic acids, sulfonic acids, sulfinic acids, phosphinic acids, etc.), bases (e.g., amine groups, proton accepting groups, etc.), or other groups that develop ionic properties when immersed in water (e.g., sulfonamides). Acryloyl groups (and to a lesser degree methacryloyl groups) and the class of acrylic polymers or polymer chains containing or terminated with oxyalkylene units (such as polyoxyethylene chains and polyoxyethylene/polyoxypropylene copolymer chains) are also well recognized as hydrophilic segments that may be present within hydrophilic polymers. Representative water-insoluble polymeric compositions are provided below, although the entire class of hydrogel materials known in the art may be used to varying degrees. The polymers set forth below and containing acidic groups can be, as an option, partially or completely neutralized with alkali metal bases, either in the monomer or the polymer, or both.

In some aspects, the first polymer comprises at least one ester-containing moiety. In some aspects, the first polymer is hydrolytically degradable, i.e., it may be hydrolytically cleaved under conditions for which the hydrogel is intended to be used. While not wishing to be bound by any particular theory, the presence of the at least one ester-containing moiety in the first polymer contributes to the observed properties of hydrolytic degradation for the first polymer provided herein. The at least one ester-containing moiety may comprise a monoester or a polyester. Representative examples of suitable polyesters include, but are not limited to, poly(lactic acid) (PLA), poly(glycolic acid) (PGA), poly(lactic-co-glycolic acid) (PLGA), and poly(ε-caprolactone) (PCL).

In some aspects, the first polymer further comprises at least one first crosslinking moiety. The at least one first crosslinking moiety may comprise any suitable functionality which can be covalently bound to the crosslinker of the hydrogels found here. Representative examples of such first crosslinking moieties include, but are not limited to, an acrylate moiety, a methyacrylate moiety, a thiol moiety, a vinyl sulfone moiety, an allyl ether moiety, a vinyl ether moiety, a maleimide moiety, a norbornene moiety, a vinyl carbonate moiety, or a fumarate moiety. In some aspects, the first crosslinking moiety may be derived from a maleimide moiety, a norbornene moiety, an acrylate moiety, a vinyl sulfone moiety, or an allyl ether moiety. In some particular aspects, the first crosslinking moiety is derived from a maleimide moiety.

In some aspects, the first polymer may comprise a polymer backbone. Some representative polymers which may comprise the polymer backbone include, but are not limited to: polyacrylic acid, polymethacrylic acid, polymaleic acid, copolymers thereof, and alkali metal and ammonium salts thereof; graft copolymers of starch and acrylic acid, starch and saponified acrylonitrile, starch and saponified ethyl acrylate, and acrylate-vinyl acetate copolymers saponified; polyvinyl alcohol, polyvinylpyrrolidone, polyvinyl alkylether, polyethylene oxide, polyacrylamide, and copolymers thereof; copolymers of maleic anhydride and alkyl vinylethers; and saponified starch graft copolymers of acrylonitrile, acrylate esters, vinyl acetate, and starch graft copolymers of acrylic acid, methacrylic acid, and maleic acid.

In some aspects, the polymer backbone may comprise a biopolymer. In some aspects, the biopolymer may have been functionalized or modified in such a manner that provides functionality enabling crosslinking with the crosslinker. Representative examples of biopolymers that may be used include, but are not limited to, collagen, gelatin, fibrin, hyaluronic acid, elastin, pectin, agarose, glycoaminoglycans, alginates, cellulose, DNA, RNA, or functionalized derivatives thereof.

In some aspects, the polymer backbone comprises a poly(ethylene glycol) or a functionalized derivative thereof.

In some aspects, the first polymer may comprise a multi-arm polymer. In such aspects, the first polymer may comprise 3 to 8 arms, for example, 3, 4, 5, 6, 7, or 8 arms. In some aspects, the first polymer comprises a core covalently bound to each arm. Representative examples of compounds from which the core may be derived include, but are not limited to, glycerol, pentaerythritol, dipentaerythritol, or tripentaerythritol.

In some aspects, the first polymer comprises a compound of Formula I:

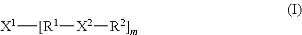

wherein:
X¹ is a core as described herein;
R¹ is a poly(ethylene glycol) backbone;
X² is the ester-containing moiety as described herein;
R² is the first crosslinking moiety as described herein; and
m is an integer selected from 3 to 8.

In some aspects of Formula I, X² comprises

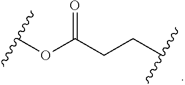 [SMILES: *CCC(=O)O*]

In some aspects of Formula I, R² comprises

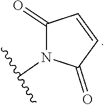 [SMILES: *N1C(=O)C=CC1=O]

In some aspects, the first polymer may have a molecular weight ranging from about 1 kDa to about 200 kDa, for example from about 1 kDa to about 175 kDa, from about 1 kDa to about 150 kDa, from about 1 kDa to about 125 kDa, from about 1 kDa to about 100 kDa, from about 1 kDa to about 75 kDa, from about 1 kDa to about 50 kDa, from about 1 kDa to about 25 kDa, from about 1 kDa to about 10 kDa, from about 1 kDa to about 5 kDa, from about 5 kDa to about 200 kDa, from about 5 kDa to about 175 kDa, from about 5 kDa to about 150 kDa, from about 5 kDa to about 125 kDa, from about 5 kDa to about 100 kDa, from about 5 kDa to about 75 kDa, from about 5 kDa to about 50 kDa, from about 5 kDa to about 25 kDa, from about 5 kDa to about 10 kDa, from about 10 kDa to about 200 kDa, from about 10 kDa to about 175 kDa, from about 10 kDa to about 150 kDa, from about 10 kDa to about 125 kDa, from about 10 kDa to about 100 kDa, from about 10 kDa to about 75 kDa, from about 10 kDa to about 50 kDa, from about 10 kDa to about 25 kDa, from about 25 kDa to about 200 kDa, from about 25 kDa to about 175 kDa, from about 25 kDa to about 150 kDa, from about 25 kDa to about 125 kDa, from about 25 kDa to about 100 kDa, from about 25 kDa to about 75 kDa, from about 25 kDa to about 50 kDa, from about 50 kDa to about 200 kDa, from about 50 kDa to about 175 kDa, from about 50 kDa to about 150 kDa, from about 50 kDa to about 125 kDa, from about 50 kDa to about 100 kDa, from about 50 kDa to about 75 kDa, from about 75 kDa to about 200 kDa, from about 75 kDa to about 175 kDa, from about 75 kDa to about 150 kDa, from about 75 kDa to about 125 kDa, from about 75 kDa to about 100 kDa, from about 100 kDa to about 200 kDa, from about 100 kDa to about 175 kDa, from about 100 kDa to about 150 kDa, from about 100 kDa to about 125 kDa, from about 125 kDa to about 200 kDa, from about 125 kDa to about 175 kDa, from about 125 kDa to about 150 kDa, from about 150 kDa to about 200 kDa, from about 150 kDa to about 175 kDa, and from about 175 kDa to about 200 kDa.

In some aspects, the second polymer comprises at least one amide-containing moiety. In some aspects, the second polymer is hydrolytically stable, i.e., it cannot be hydrolytically cleaved under the conditions for which the hydrogel is intended to be used. While not wishing to be bound by any particular theory, the presence of the at least one amide-containing moiety in the first polymer contributes to the observed properties of hydrolytic stability for the second polymer provided herein.

In some aspects, the second polymer further comprises at least one second crosslinking moiety. The at least one second crosslinking moiety may comprise any suitable functionality which can be covalently bound to the crosslinker of the hydrogels found here. Representative examples of such second crosslinking moieties include, but are not limited to, an acrylate moiety, a methyacrylate moiety, a thiol moiety, a vinyl sulfone moiety, an allyl ether moiety, a vinyl ether moiety, a maleimide moiety, a norbornene moiety, a vinyl carbonate moiety, or a fumarate moiety. In some aspects, the second crosslinking moiety may be derived from a maleimide moiety, a norbornene moiety, an acrylate moiety, a vinyl sulfone moiety, or an allyl ether moiety. In some particular aspects, the second crosslinking moiety is derived from a maleimide moiety.

In some aspects, the second polymer may comprise a polymer backbone. Some representative polymers which may comprise the polymer backbone include, but are not limited to: polyacrylic acid, polymethacrylic acid, polymaleic acid, copolymers thereof, and alkali metal and ammonium salts thereof; graft copolymers of starch and acrylic acid, starch and saponified acrylonitrile, starch and saponified ethyl acrylate, and acrylate-vinyl acetate copolymers saponified; polyvinyl alcohol, polyvinylpyrrolidone, polyvinyl alkylether, polyethylene oxide, polyacrylamide, and copolymers thereof; copolymers of maleic anhydride and alkyl vinyl ethers; and saponified starch graft copolymers of acrylonitrile, acrylate esters, vinyl acetate, and starch graft copolymers of acrylic acid, methacrylic acid, and maleic acid.

In some aspects, the polymer backbone may comprise a biopolymer. In some aspects, the biopolymer may have been functionalized or modified in such a manner that provides a functionality enabling crosslinking with the crosslinker. Representative examples of biopolymers that may be used include, but are not limited to, collagen, gelatin, fibrin, hyaluronic acid, elastin, pectin, agarose, glycoaminoglycans, alginates, cellulose, DNA, RNA, or functionalized derivatives thereof.

In some aspects, the polymer backbone comprises a poly(ethylene glycol) or a functionalized derivative thereof.

In some aspects, the second polymer may comprise a multi-arm polymer. In such aspects, the second polymer may comprise 3 to 8 arms, for example, 3, 4, 5, 6, 7, or 8 arms. In some aspects, the second polymer comprises a core covalently bound to each arm. Representative examples of compounds from which the core may be derived include, but are not limited to, glycerol, pentaerythritol, dipentaerythritol, or tripentaerythritol.

In some aspects, the second polymer comprises a compound of Formula II:

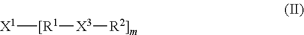

wherein:
X¹ is a core;
R¹ is a poly(ethylene glycol) backbone;
X³ is the amide-containing moiety;
R² is the first crosslinking moiety; and
m is an integer selected from 3 to 8.

In some aspects of Formula II, X³ comprises

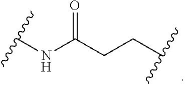 [SMILES: *CCC(=O)N*]

In some aspects of Formula II, R² comprises

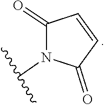 [SMILES: *N1C(=O)C=CC1=O]

In some aspects, the second polymer may have a molecular weight ranging from about 1 kDa to about 200 kDa, for example from about 1 kDa to about 175 kDa, from about 1 kDa to about 150 kDa, from about 1 kDa to about 125 kDa, from about 1 kDa to about 100 kDa, from about 1 kDa to about 75 kDa, from about 1 kDa to about 50 kDa, from about 1 kDa to about 25 kDa, from about 1 kDa to about 10 kDa, from about 1 kDa to about 5 kDa, from about 5 kDa to about 200 kDa, from about 5 kDa to about 175 kDa, from about 5 kDa to about 150 kDa, from about 5 kDa to about 125 kDa, from about 5 kDa to about 100 kDa, from about 5 kDa to about 75 kDa, from about 5 kDa to about 50 kDa, from about 5 kDa to about 25 kDa, from about 5 kDa to about 10 kDa, from about 10 kDa to about 200 kDa, from about 10 kDa to about 175 kDa, from about 10 kDa to about 150 kDa, from about 10 kDa to about 125 kDa, from about 10 kDa to about 100 kDa, from about 10 kDa to about 75 kDa, from about 10 kDa to about 50 kDa, from about 10 kDa to about 25 kDa, from about 25 kDa to about 200 kDa, from about 25 kDa to about 175 kDa, from about 25 kDa to about 150 kDa, from about 25 kDa to about 125 kDa, from about 25 kDa to about 100 kDa, from about 25 kDa to about 75 kDa, from about 25 kDa to about 50 kDa, from about 50 kDa to about 200 kDa, from about 50 kDa to about 175 kDa, from about 50 kDa to about 150 kDa, from about 50 kDa to about 125 kDa, from about 50 kDa to about 100 kDa, from about 50 kDa to about 75 kDa, from about 75 kDa to about 200 kDa, from about 75 kDa to about 175 kDa, from about 75 kDa to about 150 kDa, from about 75 kDa to about 125 kDa, from about 75 kDa to about 100 kDa, from about 100 kDa to about 200 kDa, from about 100 kDa to about 175 kDa, from about 100 kDa to about 150 kDa, from about 100 kDa to about 125 kDa, from about 125 kDa to about 200 kDa, from about 125 kDa to about 175 kDa, from about 125 kDa to about 150 kDa, from about 150 kDa to about 200 kDa, from about 150 kDa to about 175 kDa, and from about 175 kDa to about 200 kDa.

In some aspects, the crosslinker can be covalently bound to the first polymer and the second polymer via the first crosslinking moiety and the second crosslinking moiety described herein. In some aspects, the crosslinker can be covalently bound to the first crosslinking moiety by a thioether linkage. In some aspects, the crosslinker can be covalently bound to the second crosslinking moiety by a thioether linkage.

In some aspects, the crosslinker is hydrolytically degradable, i.e., it can be hydrolytically cleaved under the conditions for which the hydrogel is intended to be used. Representative examples of such crosslinkers include, but are not limited to, ethylene glycol bis(mercaptoacetate) (EGBMA); glycol di(3-mercaptopropionate); ethylene bis(thioglycolate); glyceryl dithioglycolate (GDT); and poly(ethylene glycol)-diester dithiol.

In some aspects, the crosslinker is hydrolytically stable, i.e., cannot be hydrolytically cleaved under the conditions for which the hydrogel is intended to be used. Representative examples of such crosslinkers include, but are not limited to, 1,4-dithiothreitol (DTT); poly(ethylene glycol)-dithiol (PEG-DT); and 2,2′-(ethyleneoxy) diethanethiol (EDDT).

In some aspects, the crosslinker may comprise a peptide. In some particular aspects, the crosslinker comprises a peptide that may be enzymatically (e.g., proteolytically) cleaved under the conditions for which the hydrogel is intended to be used, for example, in vivo.

Representative examples of such crosslinkers include, but are not limited to, GCRDVPMSMRGGDRCG (SEQ ID NO. 1); GCRDGDQGIAGFDRCG (SEQ ID NO. 2); GCRDGPQGIAGQDRCG (SEQ ID NO. 3); GCRDGPQGIWGQDRCG (SEQ ID NO. 4); GCRDIPESLRAGDRCG (SEQ ID NO. 5); and CVPLSLYSGC (SEQ ID NO. 6).

The above first and second polymers may be cross-linked either during polymerization or after polymerization using a first crosslinker as described herein and optionally one or more additional crosslinkers. The crosslinking may be performed using methods known to those skilled in the art, such as, for example, via initiation in the presence of radiation of via a radical initiator.

In some aspects, degradation of the hydrogel may be tunable by varying the molar ratio of the first polymer to the second polymer. In some embodiments, the molar ratio of the first polymer to the second polymer can range from about 100:1 to about 1:100, for example, about 90:1, about 80:1, about 70:1, about 60:1, about 50:1, about 40:1, about 30:1, about 25:1, about 20:1, about 15:1, about 10:1, about 5:1, about 4:1, about 3:1, about 2:1, about 1:1, about 1:2, about 1:3, about 1:4, about 1:5, about 1:10, about 1:15, about 1:20, about 1:25, about 1:30, about 1:40, about 1:50, about 1:60, about 1:70, about 1:80, about 1:90, or about 1:100. It may be readily understood to the skilled reader that a higher molar ratio of the first polymer to the second polymer would lead to increased hydrolytic degradation and shortened degradation times, while a lower molar ratio of the first polymer to the second polymer would lead to lower hydrolytic degradation and longer degradation times.

The degradation products of the hydrogel should be substantially biocompatible, i.e., will not substantially adversely affect the body, tissue, or cells of the living subject or otherwise, either at the site where the hydrogel is placed or in any other parts of the living subject. Methods for assessing the biocompatibility of a material are well known.

In some aspects, the hydrogels described herein may further comprise a payload. The payload may be encapsulated within or bound (for example, ionically or covalently bound) to the hydrogel. Representative examples of payloads include, but are not limited to, a cell, a protein, an antibody, a nucleic acid, a growth factor, a drug, a nanoparticle, a microparticle, or a fluorophore.

In some aspects, the hydrogels described herein may contain a bioactive agent capable of modulating a function and/or characteristic of a cell. For example, the bioactive agent may be capable of modulating a function and/or characteristic of a cell that is dispersed on or within the hydrogel. Alternatively or additionally, the bioactive agent may be capable of modulating a function and/or characteristic of an endogenous cell surrounding a hydrogel implanted in a tissue defect, for example, and guiding the cell into the defect. The at least one bioactive agent can include polynucleotides and/or polypeptides encoding or comprising, for example, transcription factors, differentiation factors, growth factors, or combinations thereof. The at least one bioactive agent can also include any agent capable of promoting tissue formation, destruction, and/or targeting a specific disease state (for example, cancer). Representative examples of such bioactive agents include, but are not limited to, chemotactic agents, various proteins (such as short term peptides, bone morphogenic proteins, collagen, glycoproteins, and lipoprotein), cell attachment mediators, biologically active ligands, integrin binding sequence, various growth and/or differentiation agents and fragments thereof (such as epidermal growth factor (EGF), hepatocyte growth factor (HGF), vascular endothelial growth factors (VEGF), fibroblast growth factors (e.g., bFGF), platelet derived growth factors (PDGF), insulin-like growth factor (e.g., IGF-1, IGF-II) and transforming growth factors (e.g., TGF-β I-III)), parathyroid hormone, parathyroid hormone related peptide, bone morphogenic proteins (e.g., BMP-2, BMP-4, BMP-6, BMP-7, BMP-12, BMP-13, BMP-14), transcription factors, such as sonic hedgehog, growth differentiation factors (e.g., GDF5, GDF6, GDF8), recombinant human growth factors (such as MP52 and MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, polynucleotides, DNA fragments, DNA plasmids, MMPs, TIMPs, interfering RNA molecules, such as siRNAs, oligonucleotides, proteoglycans, glycoproteins, glycosaminoglycans, and DNA encoding for shRNA.

In some embodiments, the hydrogels described herein may contain a therapeutic agent which may be used in treating a condition or disorder in a subject in need of such treatment. The term “therapeutic agent” includes any synthetic or naturally occurring biologically active compound or composition of matter which, when administered to an organism (either human or a nonhuman animal), induces a desired pharmacologic, immunogenic, and/or physiologic effect by local and/or systemic action. The term, therefore, encompasses those compounds or chemicals traditionally regarded as drugs, vaccines, and biopharmaceuticals, including molecules such as proteins, peptides, hormones, nucleic acids, gene constructs, and the like. Examples of therapeutic agents are described in well-known literature references such as the Merck Index (14^{th} Edition), the Physician's Desk Reference (64^{th} Edition), and The Pharmacological Basis of Therapeutics (12^{th} Edition), and they include, without limitation, medicaments; vitamins; mineral supplements, substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiandrenergics, antiarrhythmics, antihypertensive agents, hormones, and nutrients, antiarthritics, antiasthmatic agents, anticonvulsants, antihistamines, antinauseants, antineoplastics, antipruritics, antipyretics, antispasmodics, cardiovascular preparations (including calcium channel blockers, beta blockers, and beta-agonists), antihypertensives, diuretics, vasodilators, central nervous system stimulants, cough and cold preparations, decongestants, diagnostics, bone growth stimulants and bone resorption inhibitors, immunosuppressives, muscle relaxants, psychostimulants, sedatives, tranquilizers, proteins, peptides, and fragments thereof (whether naturally occurring, chemically synthesized or recombinantly produced), and nucleic acid molecules (polymeric forms of two or more nucleotides, either ribonucleotides (RNA) or deoxyribonucleotides (DNA) including both double and single-stranded molecules, gene constructs, expression vectors, antisense molecules and the like), small molecules and other biologically active macromolecules such as, for examples, proteins and enzymes. The agent may be a biologically active agent used in medical, including veterinary applications, and in agriculture, such as with plants, as well as other areas.

The hydrogel can be injectable and/or implantable or can be in the form of a membrane, sponge, gel, solid scaffold, spun fiber, woven or unwoven mesh, nanoparticle, microparticle, or any other desirable configuration.

In another aspect, the hydrogel can include at least one cell dispersed on or within the hydrogel. For example, cells can be entirely or partly encapsulated within the hydrogel. Cells can include, for example, any progenitor cell, such as a totipotent stem cell, a pluripotent stem cell, or a multipotent stem cell, as well as any of their lineage descendent cells, including more differentiated cells. The cells can be autologous, xenogeneic, allogeneic, and/or syngeneic. Where the cells are not autologous, it may be desirable to administer immunosuppressive agents in order to minimize immunorejection. The cells employed may be primary cells, expanded cells, or cell lines, and may be dividing or non-dividing cells. Cells may be expanded ex vivo prior to introduction into or onto the hydrogel. For example, autologous cells can be expanded in this manner if a sufficient number of viable cells cannot be harvested from the host subject. Alternatively or additionally, the cells may be pieces of tissue, including tissue that has some internal structure. The cells may be primary tissue explants and preparations thereof, cell lines (including transformed cells), or host cells.

In some embodiments, a cell can refer to any progenitor cell, such as totipotent stem cells, pluripotent stem cells, and multipotent stem cells, as well as any of their lineage descendent cells, including more differentiated cells and multicellular organoids. The terms “stem cell” and “progenitor cell” are used interchangeably herein. The cells can be derived from embryonic, fetal, or adult tissues. Examples of progenitor cells can include totipotent stem cells, multipotent stem cells, mesenchymal stromal/stem cells (MSCs), hematopoietic stem cells, neuronal stem cells, pancreatic stem cells, cardiac stem cells, embryonic stem cells, induced pluripotent stem cells, embryonic germ cells, neural crest stem cells, kidney stem cells, hepatic stem cells, lung stem cells, hemangioblast cells, and endothelial progenitor cells. Additional exemplary progenitor cells can include de-differentiated chondrogenic cells, chondrogenic cells, cord blood stem cells, multi-potent adult progenitor cells, myogenic cells, osteogenic cells, tendogenic cells, ligamentogenic cells, adipogenic cells, and dermatogenic cells.

The hydrogel can be formed with at least one cell and/or bioactive agent. For example, a plurality of cells may be dispersed in a substantially uniform manner on or within the hydrogel or, alternatively, dispersed such that different densities and/or spatial distributions of different or the same cells are dispersed within different portions of the hydrogel. The cells may be seeded before or after crosslinking of the first polymer and the second polymer. Alternatively, the hydrogel can be incubated in a solution of at least one bioactive agent after crosslinking the first polymer and the second polymer. Generally, cells can be introduced into the hydrogel in vitro or in vivo. Cells may be mixed with the hydrogel and cultured in an adequate growth (or storage) medium to ensure cell viability. If the hydrogel is to be implanted for use in vivo after in vitro seeding, for example, sufficient growth medium may be supplied to ensure cell viability during in vitro culture prior to in vivo application. Once the hydrogel has been implanted, the nutritional requirements of the cells can be met by the circulating fluids of the host subject.

Any available method may be employed to introduce the cells into the hydrogel. For example, cells may be injected into the hydrogel (such as in combination with growth medium) or may be introduced by other means, such as pressure, vacuum, osmosis, or manual mixing. Alternatively or additionally, cells may be layered on the hydrogel, or the hydrogel may be dipped into a cell suspension and allowed to remain there under conditions and for a time sufficient for the cells to incorporate within or attach to the hydrogel. Generally, it is desirable to avoid excessive manual manipulation of the cells in order to minimize cell death during the impregnation procedure. For example, in some situations, it may not be desirable to manually mix or knead the cells with the hydrogel; however, such an approach may be useful in those cases in which a sufficient number of cells will survive the procedure. Cells can also be introduced into the hydrogel in vivo simply by placing the hydrogel in the subject adjacent to a source of desired cells. Bioactive agents may be released from the hydrogel if contained therein, which may also recruit local cells, cells in the circulation, or cells at a distance from the implantation or injection site.

The number of cells introduced into the hydrogel will vary based on the intended application of the hydrogel and the type of cell used. For example, when dividing autologous cells are being introduced by injection or mixing into the hydrogel, a lower number of cells can be used. Alternatively, where non-dividing cells are being introduced by injection or mixing into the hydrogel, a larger number of cells may be required. The hydrogel may either be in a hydrated or lyophilized state prior to the addition of cells. For example, the hydrogel can be in a lyophilized state before the addition of cells is done to rehydrate and populate the hydrogel with cells.

In some aspects, the cells can be incorporated into the hydrogel, which may then be used as a cell carrier or delivery vehicle, a scaffold for tissue growth, or as an adhesive biomaterial. In some aspects, the cells incorporated into the hydrogel can include stem cells, spheroids, organoids, or other primary cells, which may be useful for therapeutic delivery.

In some aspects, the payload may comprise one or more molecules covalently bound to the hydrogel. In representative aspects, cysteine-containing peptides or proteins can be incorporated into the hydrogel structure by the appropriate thiole-ene reaction crosslinking chemistries. These molecules may be attached for any purpose suitable for the particular application, for example, cell adhesion, growth factor delivery, or protein or drug deliver.

In some aspects, the payload may comprise one or more adhesive peptides. Representative adhesive peptides which may be used include, but are not limited to, GRGDSPC (SEQ ID NO. 7), GRDGSPC (SEQ ID NO. 8), CGGRKRLQVQLSIRT (SEQ ID NO. 9), CGGEGYGEGYIGSR (SEQ ID NO. 10), CGGAASIKVAVSADR (SEQ ID NO. 11), CGGTWSQKALHHRVP (SEQ ID NO. 12), CGGAGQWHRVSVRWG (SEQ ID NO. 13), CGKKQRFRHRNRKG (SEQ ID NO. 14), CGTLFLAHGRLVFM (SEQ ID NO. 15), CGFHVAYVLIKF (SEQ ID NO. 16), CGRLVSYNGIIFFLK (SEQ ID NO. 17), CGLRRFSTAPFAFIDINDVINF (SEQ ID NO. 18), GYGGGPPGPPGPPGPPGPPGPPGFXGERPPGPPGPPGPPGPPGPC, wherein is X hydroxyproline (SEQ ID NO. 19), and X is GYGGGPPGPPGPPGPPGPPGPPGAXGERPPGPPGPPGPPGPPGPC, wherein hydroxyproline (SEQ ID NO. 20).

In some aspects, the payload may comprise a protein, a peptide, or a growth factor. Representative examples of such payloads include, but are not limited to, VEGF-A, Fas-ligand, PD-L1, IL-2, BMP-2, lysostaphin, and therapeutic antibodies.

In some aspects, the payload may comprise a fluorophore. Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs-AutoFluorescent Protein-(Quantum Biotechnologies) see sgGFP, sgBFP; Alexa Fluor 350; Alexa Fluor 430; Alexa Fluor 488; Alexa Fluor 532; Alexa Fluor 546; Alexa Fluor 568; Alexa Fluor 594; Alexa Fluor 633; Alexa Fluor 647; Alexa Fluor 660; Alexa Fluor 680; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG CBQCA; ATTO-TAG FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO-1; BOBO-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO-1; BO-PRO-3; Brilliant Sulphoflavin FF; BTC; BTC-5N; Calcein; Calcein Blue; Calcium Crimson-; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2; Cy3.1 8; Cy3.5; Cy3; Cy5.1 8; Cy5.5; Cy5; Cy7; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18 (5)); DIDS; Dihydorhodamine 123 (DHR); Dil (DilC18 (3)); I Dinitrophenol; DiO (DiOC18 (3)); DiR; DIR (DilC18 (7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43Ô; FM 4-46; Fura Red (high pH); Fura Red/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green; Oregon Green 488; Oregon Green 500; Oregon Green 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFPÔ (super glow BFP); sgGFP (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas RedÔ; Texas Red-XÔ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

The hydrogels described herein can be used in a variety of biomedical applications, including tissue engineering, drug delivery applications, and regenerative medicine. In one example, a hydrogel described herein can be used to promote tissue growth in a subject. One step of the method can include identifying a target site. The target site can comprise a tissue defect in which the promotion of new tissue is desired. The target site can also comprise a disease location (for example, a tumor). Methods for identifying tissue defects and disease locations are known in the art and can include, for example, various imaging modalities, such as CT, MRI, and X-ray. After identifying a target site, the hydrogel can be administered to the target site. Next, the hydrogel may be loaded into a syringe or other similar device and injected or implanted into the tissue defect. Upon injection or implantation into the tissue defect, the hydrogel can be formed into the shape of the tissue defect using tactile means. Alternatively, the hydrogel may be formed into a specific shape prior to implantation into the subject. After implanting, the cells can begin to migrate from the hydrogel into the tissue defect, express growth and/or differentiation factors, and/or promote cell expansion and differentiation. Additionally, the presence of the hydrogel in the tissue defect may promote the migration of endogenous cells surrounding the tissue defect into the hydrogel. Once implanted, the ester moiety can be hydrolyzed. Hydrolysis of this moiety can occur at a controlled rate and lead to controlled degradation of the hydrogel. This degradation can create space for cell growth and deposition of a new extracellular matrix to replace the hydrogel.

As used herein, the term “tissue” can refer to an aggregate of cells having substantially the same function and/or form in a multicellular organism. “Tissue” is typically an aggregate of cells of the same origin but may be an aggregate of cells of different origins. The cells can have substantially the same or substantially different functions and may be of the same or different type. “Tissue” can include, but is not limited to, an organ, a part of an organ, bone, cartilage, skin, neuron, axon, blood vessel, cornea, muscle, fascia, brain, prostate, breast, endometrium, lung, pancreas, small intestine, blood, liver, testes, ovaries, cervix, colon, stomach, esophagus, spleen, lymph node, bone marrow, kidney, peripheral blood, or embryonic tissue.

In some aspects, the hydrogels described herein can be used to deliver diverse payloads, such as cells and proteins. In such aspects, the cells can be autologous, allogeneic, or pluripotent stem cell-derived. In some aspects, larger, multicellular structures such as spheroids or organoids can be present within the gel. Cells are typically imbedded directly into the hydrogel network in such aspects. While not wishing to be bound to any theory, the hydrogels described herein typically have a nano-scale mesh structure; thus, cells are far too large to defuse or migrate out of the hydrogel network without significant degradation of the hydrogel. In some aspects, proteins present as payloads in the described hydrogels can be unmodified or modified with a terminal thiol to allow for covalent tethering. However, other chemistries are also possible. Non-limiting examples of proteins that may be used include therapeutic antibodies, VEGF, BMP-2, lysostaphin, Fas-ligand, PD-L1, TGF-beta, TNF-alpha, IL-1Ra, and IFNγ.

In some aspects, the hydrogels described herein can be used as a wound dressing. The described hydrogels can be used for delivery of cells that participate in the tissue regeneration process, including stem cells (mesenchymal stem cells, iPSC-derived tissue-specific cells, etc.), stromal cells (e.g., fibroblasts), and/or immune cells (e.g., macrophages). In addition, antimicrobial proteins/compounds can be delivered to mitigate infections. Hydrogels can also be used to deliver proteins known to promote wound healing responses. For example, VEGF-A can be delivered to promote vascularization, and bFGF can be used to promote the deposition and reorganization of extracellular matrix proteins by stromal cells. Additionally, cytokines such as IFNγ and IL-4 can be delivered to recruit and modulate the behavior of immune cell populations of interest in wound healing, especially macrophages. Temporal control over growth factor and cytokine cues in the wound repair environment are paramount. The presently disclosed hydrogels provide temporally control delivery of various proteins via tight control over the degradation of the hydrogel is a significant advantage in addressing the temporal nature of wound repair responses.

In some aspects, the hydrogels described herein can be used to generate a tolerogenic cell transplantation niche. Creating a hospitable environment for transplanted cell therapies is a significant challenge, exemplified in the field of islet transplantation for the treatment of type 1 diabetes, hepatocytes for the treatment of liver failure, or intestinal organoids to treat inflammatory bowel disorders. Transplanted cells must integrate with the host tissue, especially vascularly, in order to receive oxygen and nutrients necessary for the graft to survive. As in wound healing, delivery of vasculogenic proteins such as VEGF within the hydrogels described herein can promote de novo vascularization within the graft. Transplanted cells must be protected from attack by the host immune system, especially if the graft is allogeneic. Delivery of immunomodulatory proteins such as Fas-ligand and PD-L1 within the hydrogels described herein can reduce the insult of effector cells and promote a tolerogenic immune environment within the graft.

Kits for practicing the methods described herein are further provided. By “kit” is intended any manufacture (e.g., a package or a container) comprising at least one reagent, e.g., any one of the compositions described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

Also disclosed are kits that comprise a composition disclosed herein in one or more containers. The disclosed kits can optionally include pharmaceutically acceptable carriers and/or diluents. In one embodiment, a kit includes one or more other components, adjuncts, or adjuvants, as described herein. In one embodiment, a kit includes instructions or packaging materials that describe how to administer a composition of the kit. Containers of the kit can be of any suitable material, e.g., glass, plastic, metal, etc., and of any suitable size, shape, or configuration. In one embodiment, a composition agent disclosed herein is provided in the kit as a solid. In another embodiment, a composition disclosed herein is provided in the kit as a liquid or solution. In one embodiment, the kit comprises an ampoule or syringe containing a composition described herein in liquid or solution form.

In some aspects, the following embodiments of the present disclosure are also provided:

Embodiment 1. A hydrogel comprising:
a first polymer comprising at least one ester-containing moiety and at least one first crosslinking moiety;
a second polymer comprising at least one amide-containing moiety and at least one second crosslinking moiety; and
and a crosslinker covalently bound to the first polymer and the second polymer via the first crosslinking moiety and the second crosslinking moiety.

Embodiment 2. The hydrogel of embodiment 1, wherein the first polymer is hydrolytically degradable.

Embodiment 3. The hydrogel of embodiment 1 or embodiment 2, wherein the first crosslinking moiety is derived from a maleimide moiety, a norbornene moiety, an acrylate moiety, a vinyl sulfone moiety, or an allyl ether moiety.

Embodiment 4. The hydrogel of any one of embodiments 1-3, wherein the first crosslinking moiety is derived from a maleimide moiety.

Embodiment 5. The hydrogel of any one of embodiments 1-4, wherein the first polymer comprises a poly(ethylene glycol) backbone.

Embodiment 6. The hydrogel of any one of embodiments 1-5, wherein the first polymer is a multi-arm polymer.

Embodiment 7. The hydrogel of embodiment 6, wherein the first polymer comprises from 3 to 8 arms.

Embodiment 8. The hydrogel of embodiment 6 or embodiment 7, wherein the first polymer comprises a core covalently bound to each arm.

Embodiment 9. The hydrogel of embodiment 8, wherein the core is derived from glycerol, pentaerythritol, dipentaerythritol, or tripentaerythritol.

Embodiment 10. The hydrogel of any one of embodiments 1-9, wherein at each occurrence the ester-containing moiety comprises monoester.

Embodiment 11. The hydrogel of any one of embodiments 1-9, wherein at each occurrence the ester-containing moiety comprises a polyester.

Embodiment 12. The hydrogel of embodiment 11, wherein the polyester is selected from poly(lactic acid) (PLA), poly(glycolic acid) (PGA), poly(lactic-co-glycolic acid) (PLGA), and poly(ε-caprolactone) (PCL).

Embodiment 13. The hydrogel of any one of embodiments 1-12, wherein the first polymer comprises a compound of Formula I:

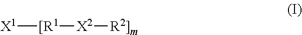

wherein:
X¹ is a core;
R¹ is a poly(ethylene glycol) backbone;
X² is the ester-containing moiety;
R² is the first crosslinking moiety; and
m is an integer selected from 3 to 8.

Embodiment 14. The hydrogel of embodiment 13, wherein X² comprises

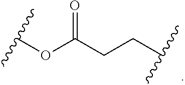 [SMILES: *CCC(=O)O*]

Embodiment 15. The hydrogel of embodiment 13 or embodiment 14, wherein R² comprises

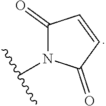 [SMILES: *N1C(=O)C=CC1=O]

Embodiment 16. The hydrogel of any one of embodiments 1-15, wherein the second polymer is hydrolytically stable.

Embodiment 17. The hydrogel of any one of embodiments 1-16, wherein the second crosslinking moiety is derived from a maleimide moiety, a norbornene moiety, an acrylate moiety, a vinyl sulfone moiety, or an allyl ether moiety.

Embodiment 18. The hydrogel of any one of embodiments 1-17, wherein the second crosslinking moiety is derived from a maleimide moiety.

Embodiment 19. The hydrogel of any one of embodiments 1-18, wherein the second polymer comprises a poly(ethylene glycol) backbone.

Embodiment 20. The hydrogel of any one of embodiments 1-19, wherein the second polymer is a multi-arm polymer.

Embodiment 21. The hydrogel of embodiment 20, wherein the second polymer comprises from 3 to 8 arms.

Embodiment 22. The hydrogel of embodiment 20 or embodiment 21, wherein the second polymer comprises a core covalently bound to each arm.

Embodiment 23. The hydrogel of embodiment 22, wherein the core is derived from glycerol, pentaerythritol, dipentaerythritol, or tripentaerythritol.

Embodiment 24. The hydrogel of any one of embodiments 1-23, wherein the second polymer comprises a compound of Formula II:

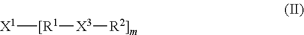

wherein:
X¹ is a core;
R¹ is a poly(ethylene glycol) backbone;
X³ is the amide-containing moiety;
R² is the first crosslinking moiety; and
m is an integer selected from 3 to 8.

Embodiment 25. The hydrogel of embodiment 24, wherein X³ comprises

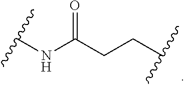 [SMILES: *CCC(=O)N*]

Embodiment 26. The hydrogel of embodiment 24 or embodiment 25, wherein R² comprises

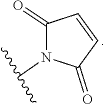 [SMILES: *N1C(=O)C=CC1=O]

Embodiment 27. The hydrogel of any one of embodiments 1-26, wherein the hydrogel has a ratio of the first polymer to the second polymer ranging from about 100:1 to about 1:100.

Embodiment 28. The hydrogel of any one of embodiments 1-27, wherein the first polymer has a molecular weight ranging from about 1 kDa to about 200 kDa.

Embodiment 29. The hydrogel of any one of embodiments 1-28, wherein the second polymer has a molecular weight ranging from about 1 kDa to about 200 kDa.

Embodiment 30. The hydrogel of any one of embodiments 1-29, wherein the crosslinker is covalently bound to the first crosslinking moiety by a thioether linkage.

Embodiment 31. The hydrogel of any one of embodiments 1-30, wherein the crosslinker is covalently bound to the second crosslinking moiety by a thioether linkage.

Embodiment 32. The hydrogel of any one of embodiments 1-31, wherein the crosslinker is hydrolytically degradable.

Embodiment 33. The hydrogel of embodiment 32, wherein the crosslinker is selected from: ethylene glycol bis(mercaptoacetate) (EGBMA); glycol di(3-mercaptopropionate); ethylene bis(thioglycolate); glyceryl dithioglycolate (GDT); and poly(ethylene glycol)-diester dithiol.

Embodiment 34. The hydrogel of any one of embodiments 1-31, wherein the crosslinker is hydrolytically stable.

Embodiment 35. The hydrogel of embodiment 34, wherein the crosslinker is selected from: 1,4-dithiothreitol (DTT); poly(ethylene glycol)-dithiol (PEG-DT); and 2,2′-(ethyleneoxy) diethanethiol (EDDT).

Embodiment 36. The hydrogel of any one of embodiments 1-31, wherein the crosslinker comprises a peptide.

Embodiment 37. The hydrogel of embodiment 36, wherein the crosslinker is selected from: GCRDVPMSMRGGDRCG (SEQ ID NO. 1); GCRDGDQGIAGFDRCG (SEQ ID NO. 2); GCRDGPQGIAGQDRCG (SEQ ID NO. 3); GCRDGPQGIWGQDRCG (SEQ ID NO. 4); GCRDIPESLRAGDRCG (SEQ ID NO. 5); and CVPLSLYSGC (SEQ ID NO. 6).

Embodiment 38. The hydrogel of any one of embodiments 1-37, further comprising a payload.

Embodiment 39. The hydrogel of embodiment 38, wherein the payload comprises a cell, a protein, an antibody, a nucleic acid, a growth factor, a drug, a nanoparticle, a microparticle, or a fluorophore.

Embodiment 40. The hydrogel of embodiment 38 or embodiment 39, wherein the payload is covalently bound to the hydrogel.

Embodiment 41. A therapeutic delivery composition comprising a hydrogel of any one of embodiments 1-40 and one or more therapeutic agents.

Embodiment 42. A cell culture medium comprising a hydrogel of any one of embodiments 1-40.

Embodiment 43. A tissue scaffold comprising a hydrogel of any one of embodiments 1-40.

Embodiment 44. A wound dressing comprising a hydrogel of any one of embodiments 1-40.

Embodiment 45. A method of delivering one or more therapeutic agents to a target site in a subject, the method comprising administering a therapeutically effective amount of a therapeutic delivery composition of embodiment 41.

Embodiment 46. The method of embodiment 45, wherein the target site is associated with a disease state or condition.

Embodiment 47. The method of embodiment 45 or embodiment 46, wherein the therapeutic delivery composition is injected or implanted into the target site.

Embodiment 48. A method of promoting tissue growth in a target site in a subject in need thereof, comprising administering to the target site a therapeutically effective amount of a hydrogel of any one of embodiments 1-40.

Embodiment 49. The method of embodiment 48, wherein the hydrogel is injected or implanted into the target site.

Embodiment 50. A method of transplanting at least one cell to a target site in a subject in need thereof, the method comprising administering to the target site an effective amount of a composition comprising the hydrogel of any one of embodiments 1-40 and the at least one cell.

Embodiment 51. The method of embodiment 50, wherein the composition is injected or implanted into the target site.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are put forth to provide those of ordinary skill in the art with a complete disclosure and description of how the compounds, compositions, articles, and methods claimed herein are made and evaluated and are intended to be purely exemplary of the invention and are not intended to limit the scope of what the inventors regard as their invention. Efforts have been made to ensure accuracy concerning numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Hydrolytic Hydrogels Tune Mesenchymal Stem Cell Persistence and Immunomodulation for Enhanced Diabetic Cutaneous Wound Healing

Diabetes is associated with an altered global inflammatory state with impaired wound healing. Mesenchymal stem/stromal cells (MSC) are being explored for treatment of diabetic cutaneous wounds due to their regenerative properties. These cells are commonly delivered by injection, but the need to prolong the retention of MSC at sites of injury has spurred the development of biomaterial-based MSC delivery vehicles. However, controlling biomaterial degradation rates in vivo remains a therapeutic-limiting challenge. In this example, we utilize hydrolytically degradable ester linkages to engineer synthetic hydrogels with tunable in vivo degradation kinetics for temporally controlled delivery of MSC. In vivo hydrogel degradation rate can be controlled by altering the ratio of ester to amide linkages in the hydrogel macromers. These hydrolytic hydrogels degrade at rates that enable unencumbered cutaneous wound healing, while enhancing the local persistence MSC compared to widely used protease-degradable hydrogels. Furthermore, hydrogel-based delivery of MSC modulates local immune responses and enhances cutaneous wound repair in diabetic mice. This example demonstrates a simple strategy for engineering tunable degradation modalities into synthetic biomaterials, overcoming a key barrier to their use as cell delivery vehicles.

### Introduction

Type 2 diabetes mellitus (T2DM) is a complex metabolic disease that affects over 30 million Americans and 400 million people worldwide [1]. Impaired wound healing is a hallmark of diabetic pathophysiology leading to diabetic foot ulcers with substantial patient morbidity. Over 100,000 lower limb amputations are performed on T2DM patients in the US every year, accounting for 75% of all non-traumatic lower limb amputations [2].

Delivery of mesenchymal stem/stromal cells (MSC) has shown beneficial effects in preclinical models of cutaneous wound healing and preliminary clinical case reports with accelerated wound closure, enhanced neovascularization, and reduced scar formation [3-7]. Although the majority of these studies were performed in healthy subjects, the potent regenerative and immunomodulatory properties of MSC may enable enhanced diabetic wound healing as well, in part by modulating wound-resident macrophage phenotypes [8, 9]. The current clinical standard for MSC therapeutic delivery is either systemic intravenous administration or local injection, which result in poor MSC persistence and engraftment at the site of injury [10]. Indeed, low MSC persistence and engraftment efficiencies plague the therapeutic application of MSC to numerous conditions, not just cutaneous wounds [11-13]. The ability of MSC to exhibit therapeutic efficacy in these studies despite their abysmal retention rates has spurred efforts to develop biomaterial-based delivery strategies for enhanced MSC persistence and engraftment to maximize MSC therapeutic potential [14]. However, whereas hydrogel delivery of cells has been shown to increase cellular persistence, current hydrogel delivery vehicles fail to meet other critical design parameters such as injectability and degradation on a relevant time scale for wound healing [14-16].

Injectable (e.g., in situ polymerizing) synthetic hydrogels generally have a nanoporous network structure with nano-scale pores that impede cell migration through the hydrogel mesh [17]. To enable tissue regeneration, these hydrogels must degrade at a sufficiently fast rate to permit tissue ingrowth. To facilitate this, hydrogels have been engineered to degrade via the incorporation of protease-degradable crosslinks [18]. However, the in vitro control over degradation rates enabled by these crosslinkers has not translated well to in vivo settings [19]. To overcome the issue of slow hydrogel degradation, various approaches have been developed to generate hydrogels with macroporous structure [20, 21]. However, specialized techniques including microfluidic generation of microgels and cryogelation are required to generate these macroporous scaffold structures [19, 22, 23]. Additionally, strategies for temporally controlled degradation of the remaining hydrogel scaffold still need to be developed.

Hydrogels containing hydrolytically degradable crosslinks are a promising strategy to overcome these issues, and proof of concept has been demonstrated in several photoinitiated polymer platforms [24, 25]. Ester-linked hydrogels are particularly appealing as they require minimal specialized techniques or equipment to fabricate and are routinely used for in vitro cell culture [26-29]. We have previously demonstrated that hydrogels with ester-linked norbornene end groups on poly(ethylene glycol) (PEG) macromers rapidly degrade in vivo via hydrolytic cleavage of the ester bond, whereas hydrogels with amide-linked end groups are largely stable in vivo [30]. However, therapeutic applications of these photopolymerized systems are limited by challenges associated with controlling light exposure in vivo.

In this example, we engineered injectable hydrogels with tunable, hydrolytic degradation rates adapted to support wound healing and promote MSC persistence and immunomodulation. We demonstrate tuning of the in vivo degradation rate of PEG hydrogels by simply altering the ratio of ester to amide linkages present in the hydrogel macromer. Hydrogels with a minimum of 50% ester linkages enable cutaneous wound closure, whereas hydrogels with only amide linkage groups significantly delay wound healing. We show that MSC persistence is increased in hydrolytic hydrogels, and that hydrolytic gel delivery of murine MSC (mMSC) accelerates wound healing in diabetic full-thickness cutaneous wounds. Finally, delivery of mMSC within hydrolytic hydrogels modulates the early immune response to full-thickness skin wounds compared to saline-delivered mMSC and cell-free controls. Together, these data demonstrate injectable hydrolytic synthetic hydrogels as improved cell delivery vehicles for regenerative medicine.

### Methods

### Hydrogel Synthesis

Amide-linked 20 kDa 4-arm PEG-4maleimide (PEG-4aMAL) with >95% purity was purchased from Laysan Bio. Custom-synthesized ester-linked 20 kDa 4-arm PEG-4maleimide (PEG-4eMAL) was synthesized by JenKem. For all studies, hydrogels were prepared with 4.0% w/v PEG-4a/eMAL macromer, 1.0 mM of the adhesive peptide GRGDSPC (RGD) (GenScript, >95% purity), and crosslinked with GCRDVPMSIMRGGDRCG (VPM) (Genscript, >95% purity). The concentration of crosslinker used for the synthesis of each hydrogel was calculated stoichiometrically by balancing the number of free thiols on the crosslinker with the number of unreacted maleimide groups on the PEG-4a/eMAL macromers following functionalization with RGD. PEG-4a/eMAL, RGD, and VPM were dissolved, individually, in 25 mM HEPES in PBS. Hydrogels were mixed at a volume ratio of 2:1:1:1 of PEG macromer: RGD: VPM: media or cell suspension. Full-thickness skin wounds each received 30 μL of hydrogel solution at a pH of ˜5.5. 50 μL of hydrogel solution at a pH of ˜4.0 was injected for IVIS studies (the lower pH prevents hydrogels from polymerizing in the syringe prior to injection; interstitial fluid buffers the hydrogel upon injection). In both cases, hydrogels polymerize in situ.

### Full-Thickness Skin Wound Surgery

All animal experiments were performed with veterinary supervision and within the guidelines of the Guide for the Care and Use of Laboratory Animals. The surgical procedure was adapted from published protocols [38, 39]. Male C57BL/6J wild-type mice (10-14 weeks old), BKS.Cg-Dock7m+/+Lepr^{db}/J obese, diabetic mice (db/db, 12-15 weeks old) and their age-matched heterozygous non-obese, non-diabetic littermate controls (db/m⁺) purchased from the Jackson Laboratories were used. The diabetic state of db/db mice was confirmed by blood glucose levels above 250 mg/dL. For gel implantation, mice were anesthetized under isoflurane, fur from the dorsal region was shaved and removed using Nair, and the skin was cleaned with 70% isopropyl alcohol and chlorohexidine. Two 4 mm diameter full-thickness wounds were created on the upper middle of the back, one on each side of the midline, using a 4 mm biopsy punch (VWR, 102096-444). The wounds were then splinted with silicone rings (6 mm inner diameter, 12 mm outer diameter) cut from a 0.5 mm thick silicone sheet (Invitrogen, P-18178) using appropriately sized biopsy punches. Silicone rings were adhered to the skin using super glue (Loctite, LOC1365882) and anchored with six interrupted 6-0 nylon sutures (eSutures, DYN9101). Wounds were then imaged on an iPhone with a ruler for scale. 30 μL hydrogels were pipetted into the wound. Wounds and hydrogels were then covered with one large piece of transparent occlusive dressing (Tegaderm). Saline conditions were injected through the wound dressing into the wound. Every other day, starting on day 3, wound dressings were removed, wounds were imaged, and new dressings were applied. Wounds were excluded if the occlusive dressing or silicone ring fell off. For cell-free hydrogel studies, an additional 30 μL of hydrogel was polymerized at the wound site during the dressing change on day 3. For studies with mMSC, the 30 μL hydrogels were used to deliver either 0, 25,000, or 250,000 mMSC to the wound on day 0 only.

### Tracking Hydrogel Degradation Via IVIS Imaging

All animal experiments were performed with veterinary supervision and within the guidelines of the Guide for the Care and Use of Laboratory Animals. Subcutaneous injections were performed as previously described [51]. Briefly, male BKS.Cg-Dock7m+/+Lepr^{db}/J obese, diabetic mice (db/db, 12-15 weeks old, Jackson Laboratories) were anesthetized under isoflurane, fur was removed from the dorsal region, and the skin was treated with 70% isopropyl alcohol and chlorohexidine. 50 μL of PEG-4a/eMAL hydrogel solution functionalized with AlexaFluor750 C5-maleimide (Invitrogen, A30459) was mixed in an Eppendorf tube on ice and then injected into the dorsal region of the mouse using a 291/2 gauge insulin syringe (Exel, 26018). Signal intensity was monitored longitudinally using an IVIS SpectrumCT imaging system (Perkin Elmer). Data were analyzed using Living Image software. Regions of interest (ROIs) were drawn around each injected hydrogel and Radiant Efficiency [p/s/sr]/[μW cm⁻²] of each ROI was quantified. The ROIs were kept the same size for all time-points. Radiant Efficiency measurements were normalized to day 0 values.

mMSC Isolation, Expansion, and Immunodepletion

All animal experiments were performed with veterinary supervision and within the guidelines of the Guide for the Care and Use of Laboratory Animals. mMSC were isolated from the bone marrow of long bones of 4-6 week old male C57BL/6J mice as described [37, 52]. Isolated cells were plated at ˜250,000 cells/cm² in a 15 cm plate in mMSC growth media (RPMI 1640, 16% MSC qualified FBS (Gibco, 12662029), 2 mM 1-glutamine, 100 U/mL pen/strep, 10 ng/mL bFGF (Austral Biologicals, GF-030-5)) and cultured overnight at 37° C., 5% O₂, 5% CO₂ (hypoxic conditions). When adherent cells reached 90-95% confluence, cells were immunodepleted by MACS separation using CD45 MicroBeads (Miltenyi Biotec, 130-052-301) and LD columns (Miltenyi Biotec, 130-042-901). Immune cell-depleted mMSC were plated at ˜2000 cells/cm² in a 15 cm plate in mMSC media containing 10 ng/mL bFGF and fed with new media every 3-4 days. mMSC were used between passages 2 and 4. mMSC were washed at least 3 times with 15 mL of PBS prior to delivery to mitigate the co-delivery of FBS which can cause a deleterious immune response [37].

Tracking mMSC Persistence Via IVIS

All animal experiments were performed with veterinary supervision and within the guidelines of the Guide for the Care and Use of Laboratory Animals. mMSC were isolated from the bone marrow of B6; FVB-Ptprc^{a} Tg(CAG-luc,-GFP)L2G85Chco Thy1^{a}/J mice (B6 Cag-luc, 4-6 weeks old, Jackson Laboratories), expanded, and immunodepleted as described above (mMSC^{luc}). Subcutaneous injections were performed as previously described [51]. Male BKS.Cg-Dock7m+/+Lepr^{db}/J obese, diabetic mice (db/db, 12-15 weeks old, Jackson Laboratories) were anesthetized under isoflurane, fur was removed from the dorsal region, and the skin was treated with 70% isopropyl alcohol and chlorohexidine. 50 μL of PEG-4a/eMAL hydrogel solution containing 150,000 mMSC^{luc} was mixed in an Eppendorf tube on ice and then injected into the dorsal region of the mouse using a 291/2 gauge insulin syringe (Exel, 26018). Saline-delivered mMSC^{luc} were injected with 50 μL of saline. Bioluminescence of transplanted hMSC^{FLuc} was measured using an IVIS Spectrum CT (Perkin Elmer). Luciferin salt (Promega) was dissolved in physiological saline and sterile filtered through 0.22 μm pore membranes. Mice received a 150 mg/kg luciferin dose injected into the intraperitoneal cavity. Time to peak signal intensity was determined for each time point and 2D bioluminescence images were acquired 10-60 min post injection and analyzed with Living Image software (Perkin Elmer).

### Histology

Following euthanasia at day 11 post-wounding and hydrogel delivery, wounds and the surrounding soft tissue were explanted and fixed in 10% formalin solution. Tissue was embedded in paraffin, sectioned, and stained with H&E. To characterize wound healing quality, epidermal thickness was measured on the H&E stained sections using ImageJ as previously described [53]. For each wound, epidermal thickness was measured at 5 different points across the length of the wound. 3 independent wounds were measured per experimental group. To determine the average epidermal thickness, for each wound, the epidermal thickness was averaged. These values were then averaged across all wounds in the treatment group.

### In Vivo Immune Profiling Via Spectral Flow Cytometry

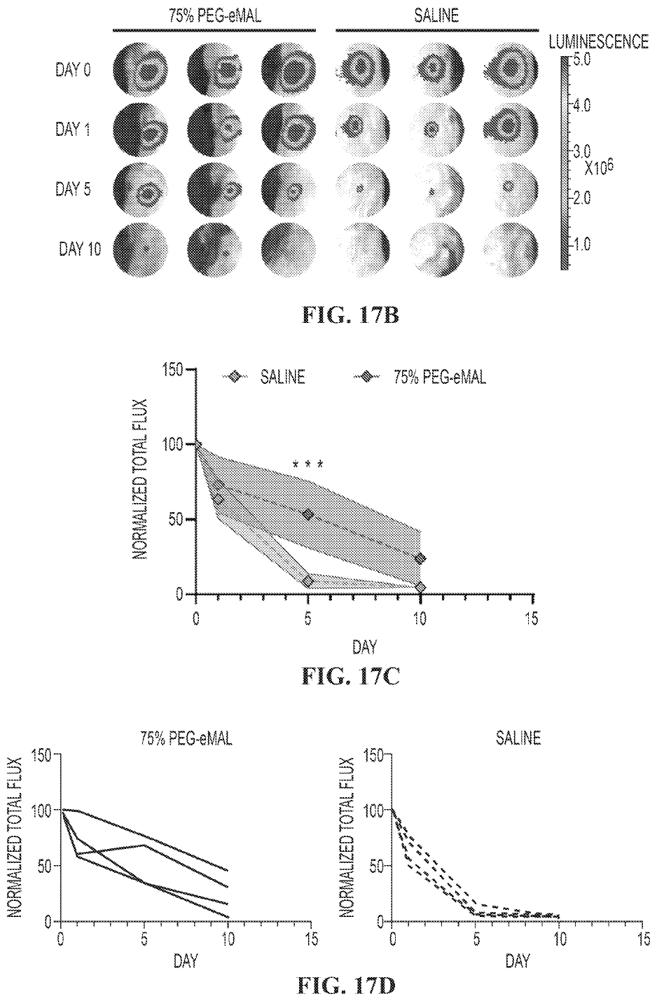

Full-thickness skin wounds were made in diabetic, obese db/db mice as described above and 75% PEG-4eMAL hydrogels with or without 250,000 mMSC were pipetted into the wound and allowed to polymerize in situ on day 0. Day 0 injections of saline at the wound site, with or without 250,000 mMSC, served as controls. On day 3 post-treatment, mice were sacrificed and the wounds and surrounding dermal tissue were explanted using a 12 mm biopsy punch. Samples were finely chopped and digested using dispase II and type II collagenase dissociation buffer (RPMI 1640, 2.5 U/mL Dispase II (ThermoFisher, 17105041), 0.2% Type II Collagenase (Worthington Biochemical Corp., LS004176)) at 37° C. for 1 h. The digested tissue suspension was then passed through a 35 μm cell strainer (Falcon, 352235) and washed with 5 mL of PBS. Red blood cells were lysed using RBC Lysis Buffer (BioLegend, 420301) and the cell pellet was rinsed first with FACS buffer (HBSS (ThermoFisher, 14175103), 2 mM EDTA (Invitrogen, AM9260G), 0.5% BSA (Sigma-Aldrich, A9418)) then with PBS. Cells were stained with Fixable Blue Dead Cell Dye (ThermoFisher, L23105) for 15 min, washed with FACS buffer, and blocked with TruStain FcX PLUS (BioLegend, 156604) prior to staining with the spectral flow cytometry panel (Table 1). Cells were fixed using Fixation Buffer (BioLegend, 420801). Absolute cell counts were determined using Precision Count Beads (BioLegend, 424902). Data was collected using a Cytek Aurora spectral flow cytometer. FCS files were imported into FCSExpress (De Novo Software) and the CD45⁺CD11b⁺ myeloid population was identified using bivariate plots (FIG. 17A). The myeloid population was then exported from FCSExpress and imported into R for further analysis using the CATALYST pipeline [54]. High-dimensional clustering was achieved by implementing FlowSOM. The FlowSOM identified clusters were then visualized in two-dimensional space using the tSNE algorithm. The number of cells in each FlowSOM-identified cluster and the median fluorescence intensity (MFI) values for markers of interest within each cluster were exported from R and graphed using GraphPad Prism 9.

TABLE 1
Spectral Flow Cytometry Panel

| | | | Catalog |
|---|---|---|---|
| Marker | Fluorochrome | Company | Number |
| Live/Dead | Fixable Blue | ThermoFisher | L23105 |
| | Dead Cell Dye | | |
| Siglec-F | BUV395 | BD Biosciences | 740280 |
| MHC-II (I-A/I-E) | BUV661 | BD Biosciences | 750280 |
| Ly6G | BUV737 | BD Biosciences | 741813 |
| CD9 | BUV805 | BD Biosciences | 741954 |
| MerTK | BV421 | Biolegend | 151510 |
| CD19 | BV480 | BD Bioscience | 566107 |
| CD8a | BV570 | Biolegend | 100740 |
| CD86 | BV605 | Biolegend | 105125 |
| NK1.1 | BV650 | Biolegend | 108736 |
| Ly6C | BV711 | Biolegend | 128037 |
| CD11c | BV785 | Biolegend | 117336 |
| F4/80 | AF488 | Biolegend | 123120 |
| CD4 | Spark Blue 550 | Biolegend | 100474 |
| CD45 | PerCP | Biolegend | 103130 |
| CD11b | PerCP-Cy5.5 | Biolegend | 101228 |
| CD3 | PE Dazzle 594 | Biolegend | 100246 |
| CD80 | PE-Cy5 | Biolegend | 104712 |
| CD301b | APC | Biolegend | 146814 |
| CD206 | AF700 | Biolegend | 141734 |
| CD163 | APC Fire 810 | Biolegend | 155321 |

### Statistical Methods

All statistical analysis was performed using GraphPad Prism 9 software. Data are plotted as mean±SD for all bar graphs. For graphs plotted as a function of time, the points at each timepoint represent the mean and the shaded region represents the SD. Figure legends indicate the specific tests run on each data set, including one-way or repeated-measures ANOVAs with indicated multiple comparisons tests. A nested ANOVA (5 readings per wound, nested within subject) was used for analysis of epithelial thickness measurements. Half-life analysis was calculated using one phase decay, least squares fit for each individual sample and the resulting half-life values were plotted in a bar graph. Statistical significance is defined as *p<0.05, ** p<0.01, *** p<0.001, **** p<0.0001 throughout the paper.

### Hydrogel Rheology

Rheology measurements were performed as previously described [55]. Briefly, 10 μL hydrogels were cast in vitro and allowed to swell in PBS overnight at 4° C. Rheological measurements were made using a cone and plate rheometer (MCR302, Anton Paar). The hydrogel sample was loaded onto the plate, domed surface up. The cone was lowered, excess hydrogel was trimmed, and PBS was added to keep the hydrogel fully swollen for the duration of the test. The storage modulus was measured over a range of angular frequencies at a strain determined to be within the viscoelastic region for the gels.

### Hydrogel Mass Swelling Ratio

50 μL of 100% PEG-4aMAL hydrogel solution was injected the dorsal subcutaneous space of male C57BL/6J wild-type mice (10-14 weeks old) and allowed to polymerize in situ. Hydrogels were then allowed to swell in situ for 4 hours prior to euthanasia of the day 0 mice. Day 28 mice were sacrificed at that time point. Following euthanasia, hydrogels were explanted and carefully cut away from any adherent soft tissue. Wet mass of the hydrogel was recorded immediately upon takedown. Hydrogels were then snap frozen and lyophilized for 24 hours. The mass of the lyophilized hydrogels was recorded as the dry mass. The mass swelling ratio was calculated as:

$$Swelling ratio = \frac{{M}_{w} - {M}_{d}}{{M}_{d}}$$

where M_{w} is the wet mass of the hydrogel and M_{d} is the dry mass.
   mMSC Phenotyping by Flow Cytometry

mMSC surface marker expression analysis was performed using a BD FACS Aria III flow cytometer (BD Biosciences). mMSC were analyzed for the markers in Table 2.

TABLE 2
mMSC Phenotyping Flow Cytometry Panel

| | | | | Catalog |
|---|---|---|---|---|
| Marker | Fluorochrome | Isotype | Company | Number |
| CD31 | PE-Cy7 | Rat IgG2a, kappa | Biolegend | 102471 |
| CD34 | PE-Cy7 | Armenian Hamster | Biolegend | 128617 |
| CD45 | FITC | Rat IgG2b, kappa | Biolegend | 157213 |
| CD44 | FITC | Rat IgG2b, kappa | Biolegend | 103005 |
| Sca-1 | PE-Cy7 | Rat IgG2a, kappa | Biolegend | 108113 |
| CD105 | PE-Cy7 | Rat IgG2a, kappa | Biolegend | 120409 |

mMSC Osteogenesis Assay

mMSC were seeded at 6000 cells/cm² in a 6-well plate and cultured in growth media under normoxic conditions until they reached 70-80% confluence. mMSC were then swapped to osteogenic differentiation media (mMSC growth media, 10 nM dexamethasone, 20 mM sodium β-glycerophosphate, 50 μM L-ascorbic acid 2-phosphate). Media was changed every 3-4 days until the end of the experiment. After 14 days of osteogenic culture, matrix mineralization was assessed by Alizarin red staining for calcium deposits.

### Results

Incorporation of Ester Linkages within PEG-4MAL Macromers Tunes Hydrogel Degradation Rates

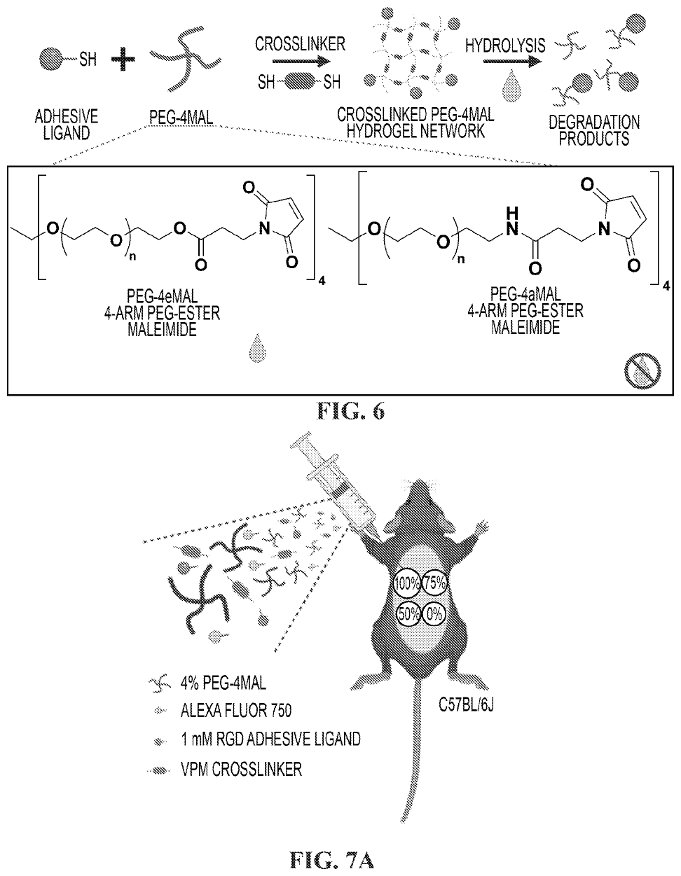

Hydrogels based on multi-arm poly(ethylene glycol) (PEG) macromers functionalized with maleimide end groups (such as 4-arm macromers, PEG-4MAL) are a versatile synthetic biomaterial platform suitable for use in a variety of biomedical applications. The thiol-maleimide reaction used to polymerize these hydrogels outperforms free-radical polymerization and other Michael-type addition chemistries in generating structurally-defined hydrogels with stoichiometric incorporation of bioligands, tunable mechanics, improved crosslinking efficiency, injectability, and excellent cytocompatibility [31, 32]. Furthermore, PEG-4MAL exhibits minimal inflammation and toxicity in vivo and is rapidly excreted in the urine, important considerations for clinical applications [33]. To explore whether modulation of the linkage group chemistry could be used to control in vivo degradation rates in bulk PEG-4MAL gels, we synthesized 4-arm PEG macromers (20 kDa) with either an amide (PEG-4aMAL) or ester (PEG-4eMAL) linkage group preceding the maleimide end group (FIG. 6). Hydrogels were prepared by reacting 4% w/v of PEG-4a/eMAL macromer with 1.0 mM of the cysteine-terminated cell adhesion peptide RGD and the proteolytically-degradable crosslinker VPM.

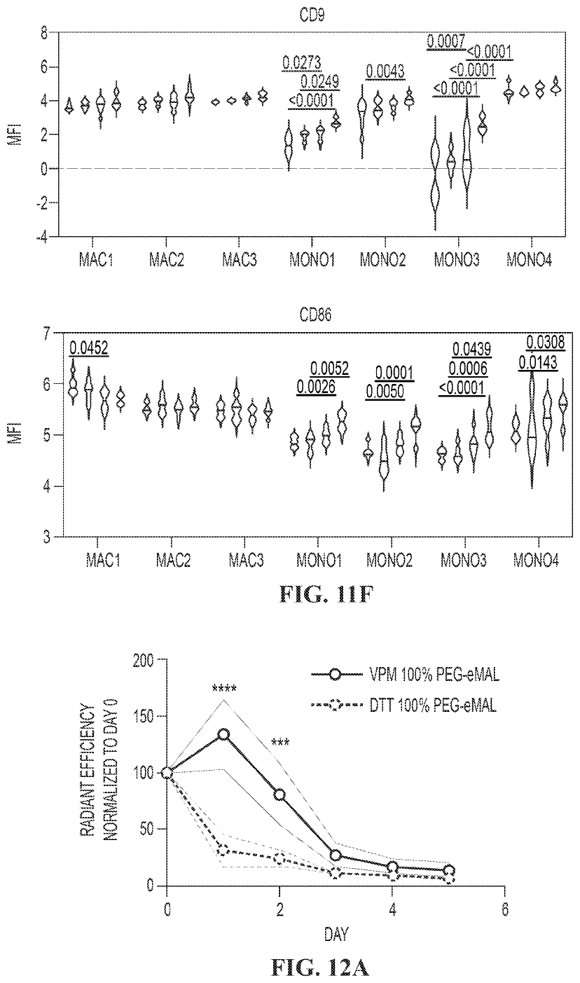

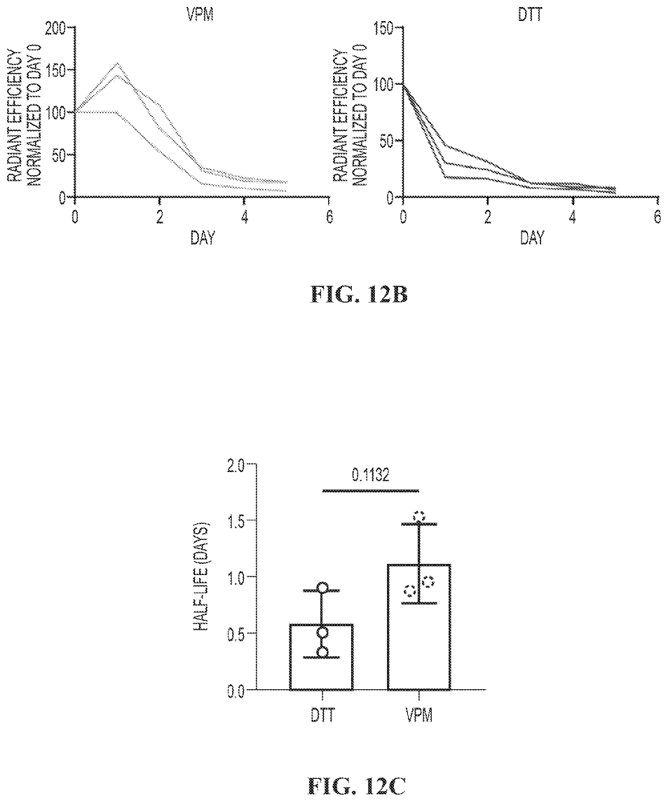

We used VPM as the crosslinker as VPM-crosslinked PEG-4aMAL hydrogels are frequently used in cell and protein delivery applications in vivo [11, 34-36]. The robust proteolytic degradation of VPM in vitro is often conflated with a similar degradation capacity in vivo; however, we and others [19, 20] have shown the degradation kinetics of VPM to be greatly reduced in vivo. Nevertheless, we use these VPM-crosslinked PEG-4aMAL hydrogels as a reference in this example. Additionally, and counterintuitively, the VPM crosslinker retards ester-linked PEG hydrogel hydrolysis in vivo compared to the non-degradable crosslinker DTT (FIGS. 12A-12B), with mean half-lives of 0.6 and 1.1 days for the DTT-crosslinked and VPM-crosslinked PEG-4eMAL hydrogels respectively (FIG. 12C).

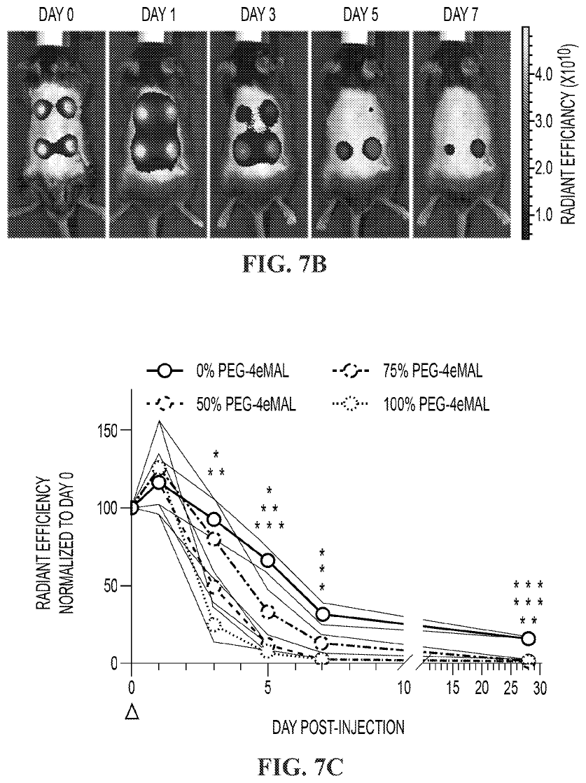

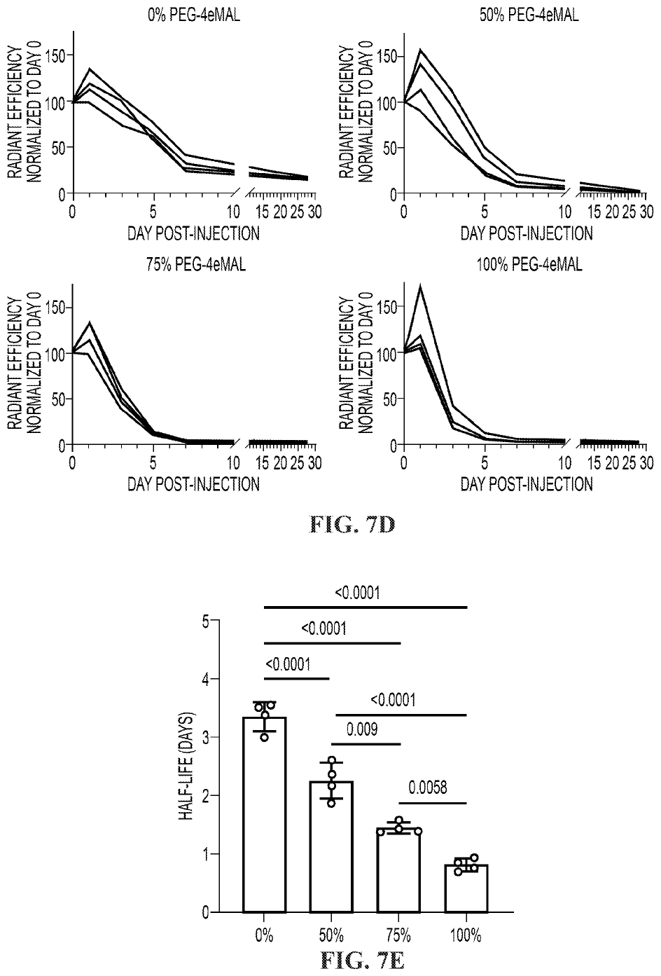

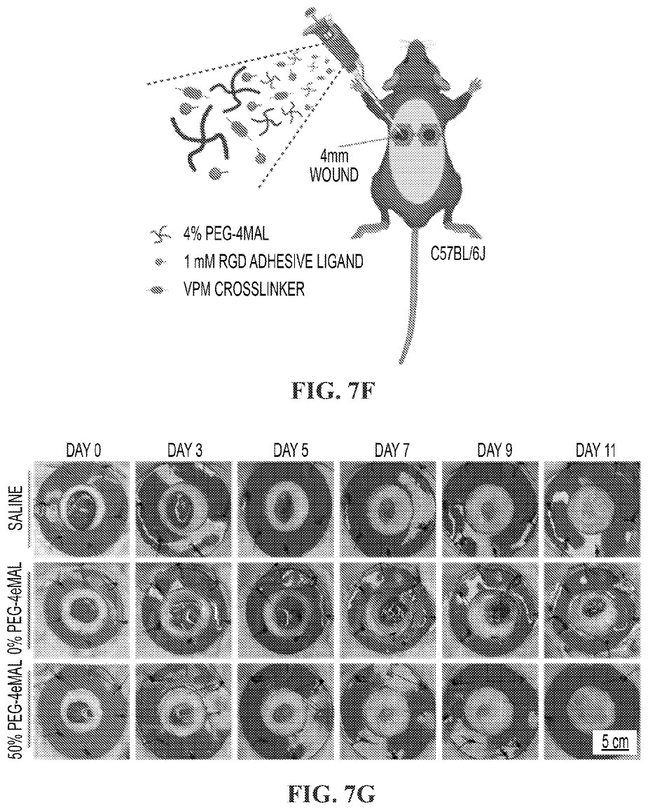

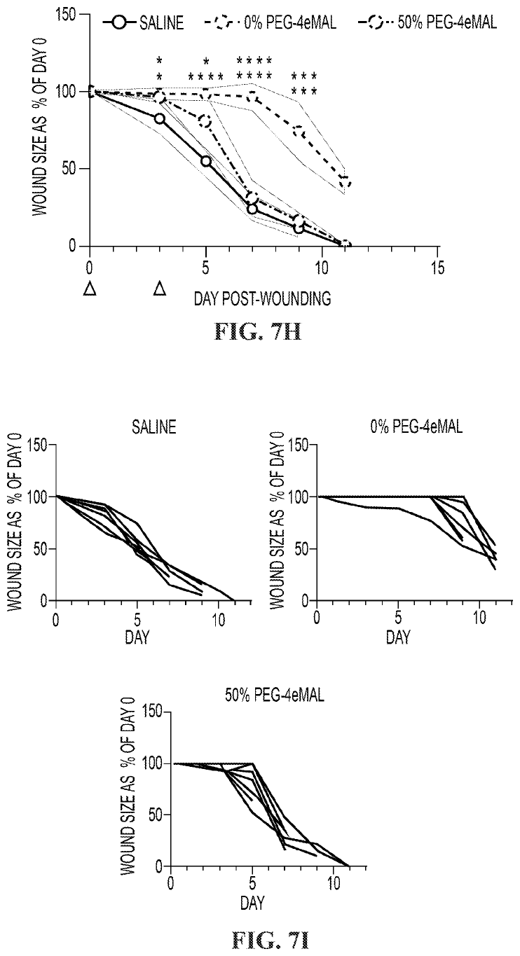

We previously showed that PEG-norbornene hydrogels containing 100% ester linkers rapidly degrade in vivo [30]. Therefore, we hypothesized that by changing the ratio of ester linker to amide linker within the hydrogel we would be able to tune the in vivo degradation rate. Control of ester linkage composition was accomplished by varying the ratio of ester- to amide-containing macromer while keeping the polymer density constant at 4%. We compared four different PEG-4eMAL to PEG-4aMAL macromer ratios: 100% PEG-4aMAL (0% PEG-4eMAL), 50/50 PEG-4eMAL/PEG-4aMAL (50% PEG-4eMAL), 75/25 PEG-4eMAL/PEG-4aMAL (75% PEG-4eMAL), and 100% PEG-4eMAL. 50 μL of PEG-4MAL hydrogel solution was injected and polymerized in situ into each of the subcutaneous dorsal quadrants of C57BL/6J mice (FIG. 7A). We have previously demonstrated minimal immunological crosstalk between the subcutaneous dorsal quadrants in this model [37], enabling us to inject all four experimental groups into the same mouse to reduce inter-subject variance. An Alexa Fluor 750 dye with a maleimide end group was tethered into the gel to enable hydrogel tracking via IVIS imaging, and the subjects were imaged every other day.

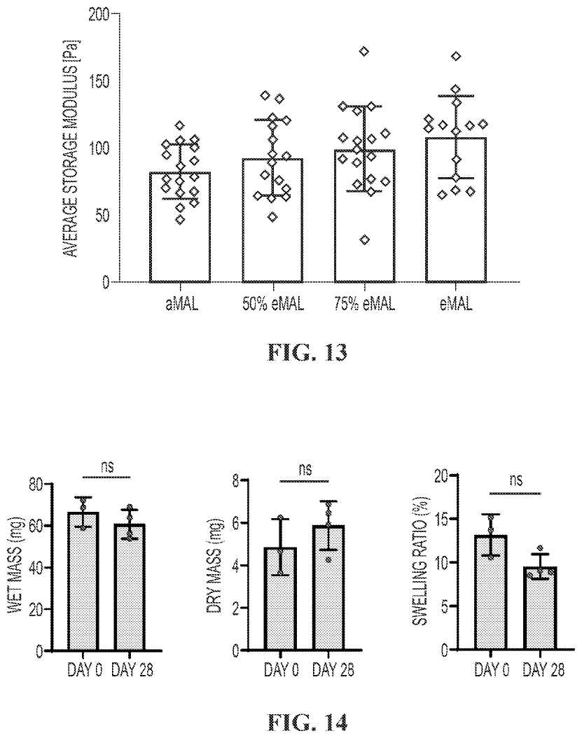

By day 3, the gels containing 75% and 100% PEG-4eMAL had significantly lower fluorescence signal than the 50% PEG-4eMAL and 0% PEG-4eMAL (100% PEG-4aMAL) gels (FIGS. 7B-7D). All of the PEG-4eMAL containing hydrogels had significantly lower signal than 0% PEG-4eMAL (100% PEG-4aMAL) gels by day 5. Furthermore, the hydrogel half-life decreased with increasing proportion of ester linker (FIG. 7E), with mean half-lives of 3.4, 2.3, 1.4, and 0.8 days for the 0%, 50%, 75%, and 100% PEG-4eMAL hydrogels, respectively. Importantly, the different gel formulations have equivalent mechanical properties upon polymerization in vitro (FIG. 13), indicating equivalent initial network architectures.

The fluorescence signal for PEG-4aMAL hydrogels steadily decreases over time, but we posit that this is primarily due to degradation of the dye and pigmentation of the mouse skin upon hair regrowth, rather than due to gel degradation. To test this, we injected 0% PEG-4eMAL (100% PEG-4aMAL) hydrogels subcutaneously in C57BL/6J mice and explanted the gels either after polymerization and swelling on day 0 or after 28 days in vivo. We assessed the explanted hydrogel wet mass, dry mass, and swelling ratio at both time points (FIG. 14). There was no difference between pre- and post-implantation values for any of these measurements indicating that the hydrogels are not appreciably degrading during this time frame despite containing the proteolytically degradable crosslinker VPM. Thus, the differences in degradation kinetics are directly related to the ester linker content of the hydrogels, and hydrogels containing no ester linkers degrade minimally in vivo.

Taken together, this data demonstrates that incorporation of ester linkages into the PEG macromer enables tunable hydrogel degradation rates without altering other physical properties of the hydrogel such as polymer weight percentage or crosslink density.

### Ester-Linked PEG-4MAL Hydrogels Enable Cutaneous Wound Closure

We examined the ability of cutaneous wounds to heal following delivery of different hydrogel formulations. Full-thickness skin wounds were generated in the dorsal region of C57BL/6J mice using a 4 mm biopsy punch (FIG. 7F). The wounds were splinted open with silicone rings to prevent wound healing by contraction, allowing only wound healing by re-epithelialization which is more similar to the wound healing process of human skin [38]. Following splinting, 0% PEG-4eMAL (100% PEG-4aMAL) hydrogel solution, 50% PEG-4eMAL hydrogel solution, or saline was pipetted into the skin wounds and allowed to polymerize in situ. A second application of hydrogel/saline was delivered to the wound on day 3.

50% PEG-4eMAL hydrogels readily degraded upon delivery to the cutaneous wounds over the first 3 days, consistent with the hydrogel degradation kinetics in the IVIS study. Delivery of these hydrolytically degradable gels resulted in wound healing times similar to that of the saline controls (FIG. 7G-7I). In contrast, PEG-4aMAL hydrogels failed to degrade rapidly enough to allow for uninhibited cutaneous wound healing. On day 7, the PEG-4aMAL hydrogels were still present at the wound site in most mice (FIG. 7G). Between the day 7 and day 9 time points, the PEG-4aMAL hydrogels dried out and/or peeled off with the Tegaderm dressing, and, once removed, the wounds were able to heal.

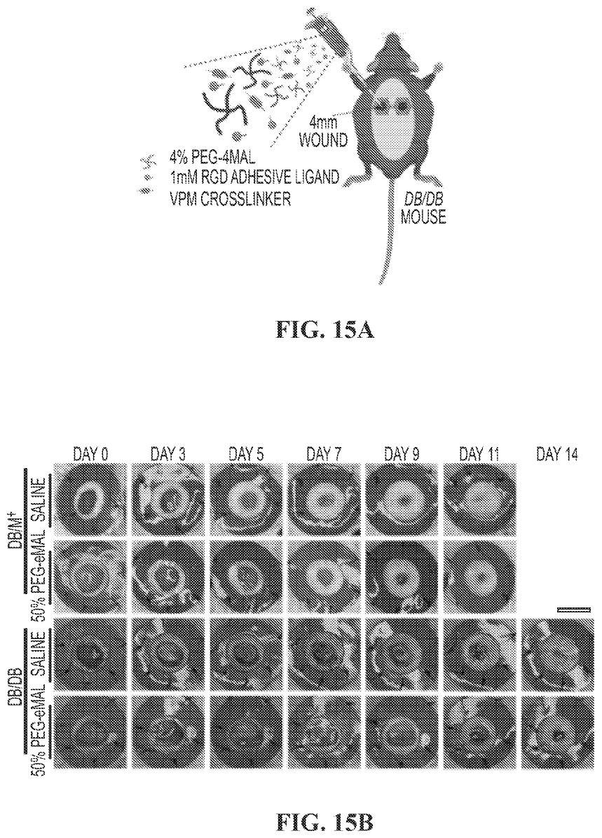

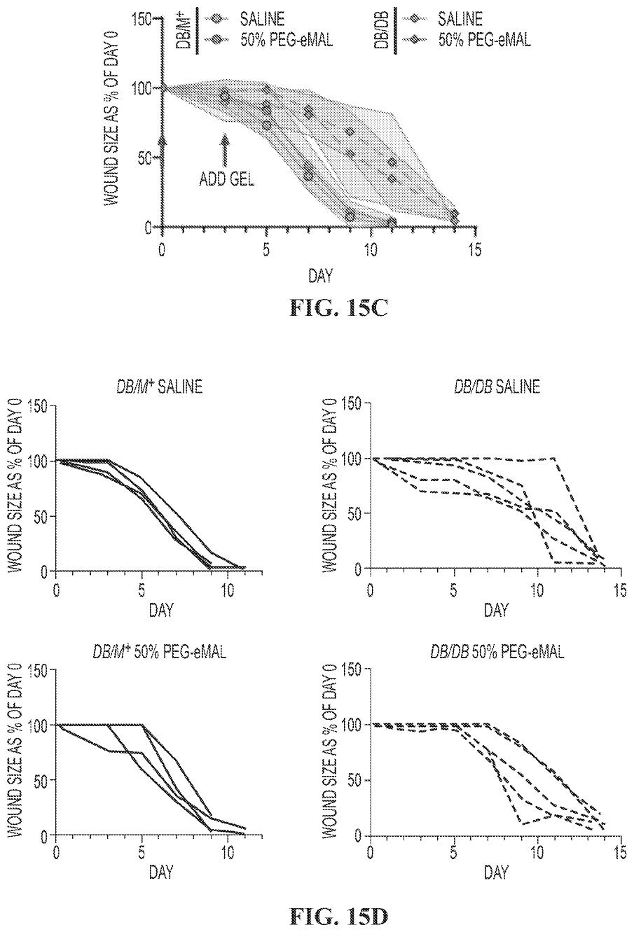

We next assessed whether ester-linked hydrogels would similarly enable wound closure in the obese, type 2 diabetic db/db mouse model. 4 mm full-thickness skin wounds were created in the dorsal region of diabetic (blood glucose >250 mg/dL) db/db mice and their age-matched heterozygous non-obese, non-diabetic littermates (db/m⁺) (FIG. 15A). Consistent with the literature [39, 40], cutaneous wound healing was significantly impaired in db/db mice compared to db/m⁺ mice (FIGS. 15B-15E). The addition of 50% PEG-4eMAL hydrogels to the wounds on days 0 and 3 did not impair wound repair compared to saline controls in either db/db mice or db/m⁺ mice, consistent with the results seen in C57BL/6J mice. Although it did not affect the wound healing rate, 50% PEG-4eMAL hydrogels were still visually present in db/db wounds at day 7, whereas they had fully degraded in db/m⁺ controls at that time point (FIG. 15B), suggesting that ester bonds may hydrolyze more slowly in diabetic mice than in non-diabetic control mice.

Taken together, these results show that non-hydrolytic amide-linked PEG-4MAL hydrogels (100% PEG-4aMAL) impede cutaneous wound repair. Modulating the degradation rate of these gels by ester incorporation enables rapid wound repair on the same time scale as control, saline-injected wounds in both healthy and diabetic mice, supporting the application of hydrolytic ester-linked gels as cell and/or drug delivery vehicles for cutaneous wound repair.

### Hydrogel Ester Content Tunes Degradation in Diabetic Mice

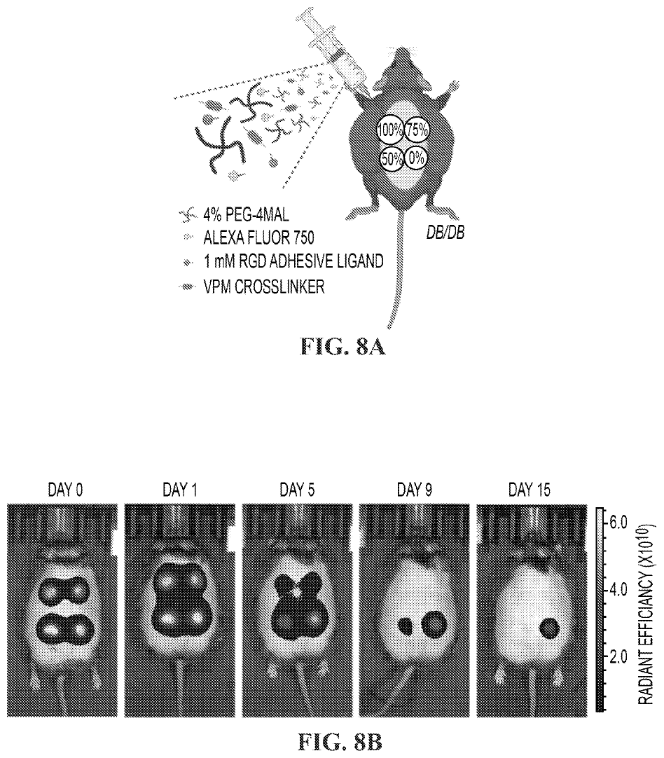

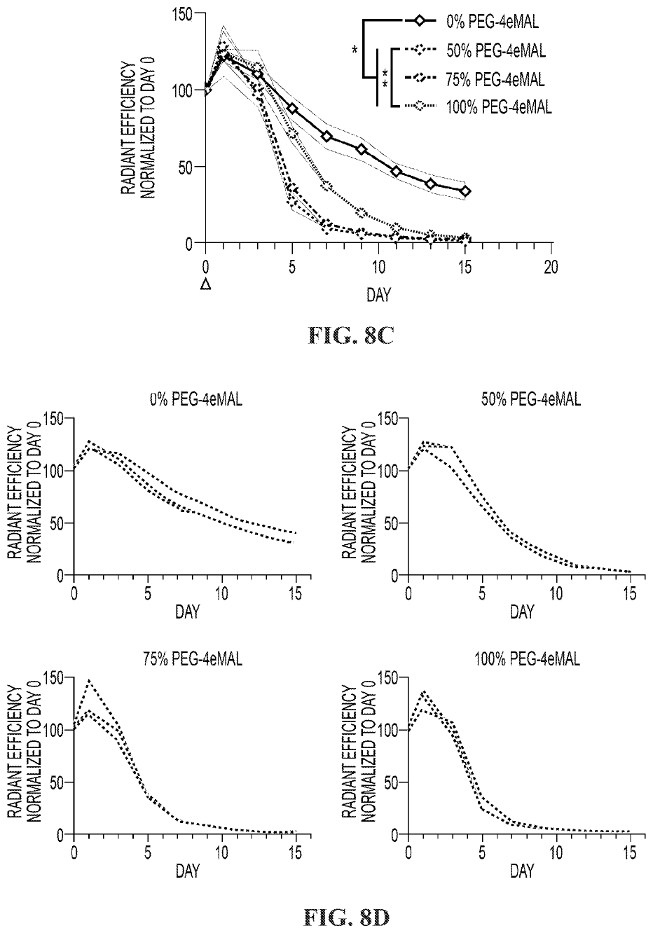

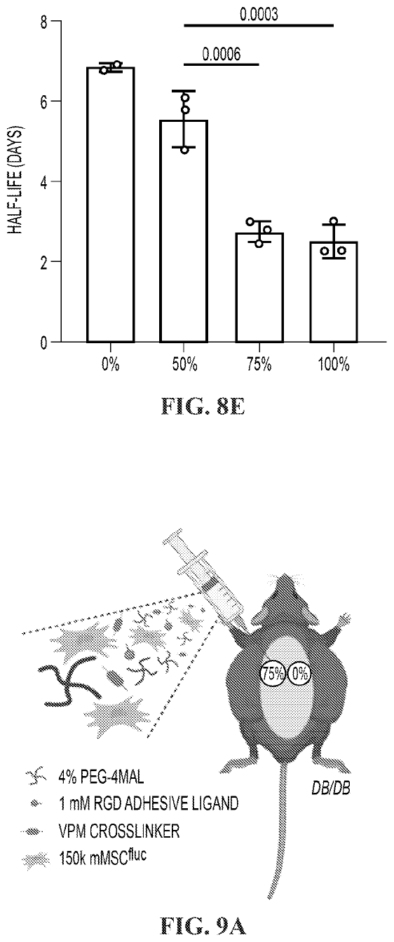

We next explored whether PEG hydrogel in vivo degradation rates in diabetic mice can be tuned by varying the proportion of ester and amide linkers in the gel. To determine their degradation kinetics, PEG hydrogels were tagged with AlexaFluor 750, injected subcutaneously in the dorsal region of db/db mice, and tracked via in vivo fluorescence imaging (IVIS). Hydrogels with 0%, 50%, 75%, and 100% PEG-4eMAL macromers as a percentage of total PEG-4MAL macromers were evaluated. 75% and 100% PEG-4eMAL gels exhibited similar degradation profiles and degraded the fastest with mean calculated half-lives of 2.8 and 2.5 days respectively (FIGS. 8A-8E). 50% PEG-4eMAL hydrogels degraded slower with a mean half-life of 5.6 days. The ˜5 day half-life of this gel corresponds with our observations in the non-diabetic C57BL/6J wound model, where the 50% gels persisted for at least 4 days post-delivery (FIG. 7C).

The amide-linked, non-hydrolytic PEG-4aMAL hydrogels (0% PEG-4eMAL) degraded the slowest (FIGS. 8C-8D). There was a slow decrease in fluorescence signal over time that can be attributed to slow degradation of the hydrogel and dye over time. Unlike C57BL/6J mice, db/db mice did not regrow hair during the 15 day study, so regrowth of highly pigmented hair was likely not a significant factor in signal loss over time here. Going forward, we chose to use the 75% PEG-4eMAL hydrogels for cell delivery studies in order to maximize the retention time of hydrogel-delivered mMSC at the wound site, while minimizing the negative impact of overly slow hydrogel degradation rates on wound healing.

Hydrolytic Hydrogels Enhance mMSC Persistence at the Site of Injection

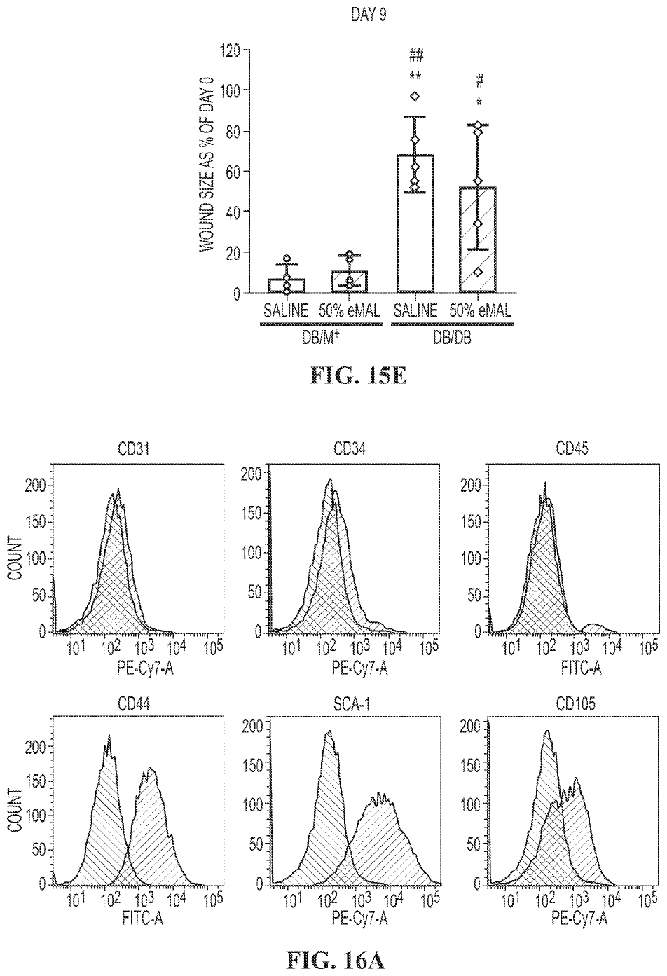

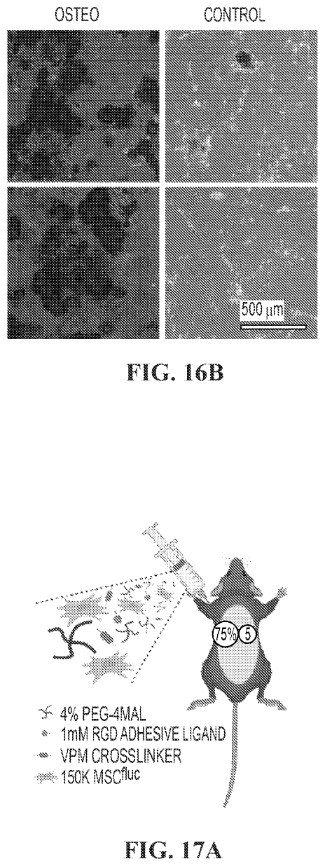

We explored ester-linked, hydrolytic hydrogels for use as therapeutic cell delivery vehicles. As previous literature has shown that bolus injected cells typically do not persist at the site of injection [15, 41, 42], we sought to evaluate whether encapsulation within hydrolytic hydrogels enhances murine MSC (mMSC) persistence. Luciferase-expressing mMSC (mMSC^{fluc}) were isolated from the bone marrow of mice harboring the CAG-luc-eGFP L2G85 transgene. Cells from these mice exhibit widespread expression of luciferase driven by the CAG promoter. mMSC^{fluc} were phenotypically characterized by flow cytometry for expression of mMSC positive markers (CD44, Sca-1, CD105) and lack of expression of mMSC negative markers (CD31, CD34, CD45) (FIG. 16A). Additionally, mMSC^{fluc} were functionally characterized for their ability to undergo osteogenic differentiation (FIG. 16B) prior to use in further studies. Isolated and expanded mMSC^{fluc} were then injected into the subcutaneous space of C57BL/6J mice in either hydrolytic 75% PEG-4eMAL hydrogels or saline and tracked over time via IVIS bioluminescence imaging (FIG. 17A). Consistent with previous literature, the bioluminescence signal from mMSC^{fluc} injected in saline rapidly decreased over the first 5 days following injection (FIGS. 17B-17D). In contrast, mMSC^{fluc} injected in 75% PEG-4eMAL hydrogels maintained a significantly higher bioluminescence signal after injection compared to saline-injected cells (FIGS. 17B-17D), despite the fact that 75% PEG-4eMAL hydrogels have largely degraded after five days in C57BL/6J mice (FIGS. 7B-7D). This result indicates that hydrolytic hydrogels facilitate cell engraftment into the local tissue, and that the persistence of the hydrogels themselves is not necessary for continued persistence of the cells after a certain point.

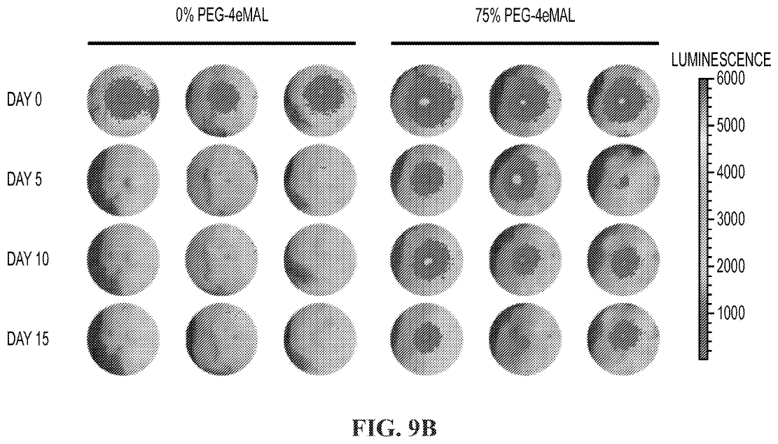

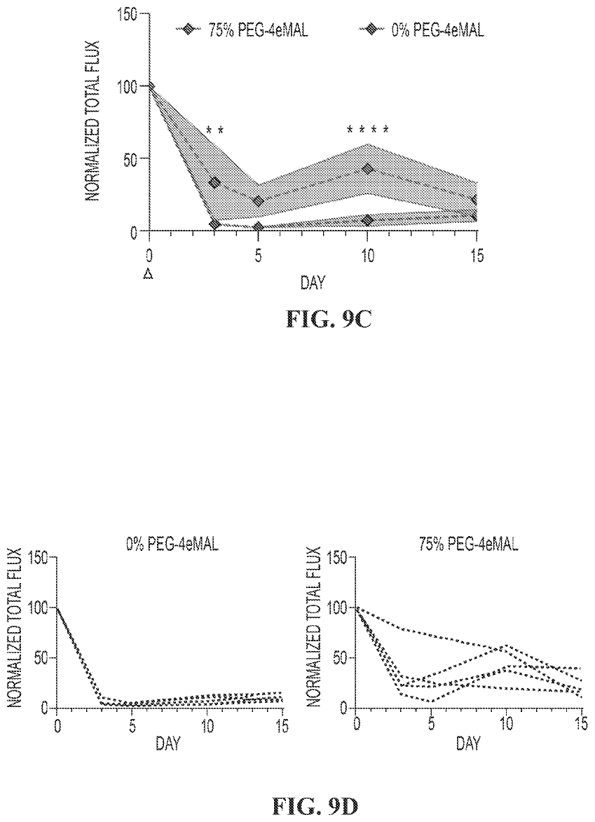

To explore whether hydrogel formulation impacts mMSC persistence in diabetic mice, mMSC^{fluc} were injected into the subcutaneous space of db/db mice in either proteolytic, non-hydrolytic 0% PEG-4eMAL (100% PEG-4aMAL) or hydrolytic 75% PEG-4eMAL hydrogels and tracked over time via IVIS bioluminescence imaging (FIGS. 9A-9D). At several time points, the bioluminescence signal for mMSC^{fluc} was significantly higher in the 75% PEG-4eMAL gels compared to the 0% PEG-4eMAL gels (FIG. 9C). We posit that as the hydrolytic hydrogels degrade, there is improved nutrient transport into the gels at earlier time points and increased ability of the cells to migrate out and engraft at later time points. Taken together, these studies show that hydrolytic hydrogels result in enhanced mMSC persistence when compared to bolus delivery in saline and delivery in non-hydrolytic PEG hydrogels.

Hydrogel Delivery of mMSC Accelerates Cutaneous Wound Healing

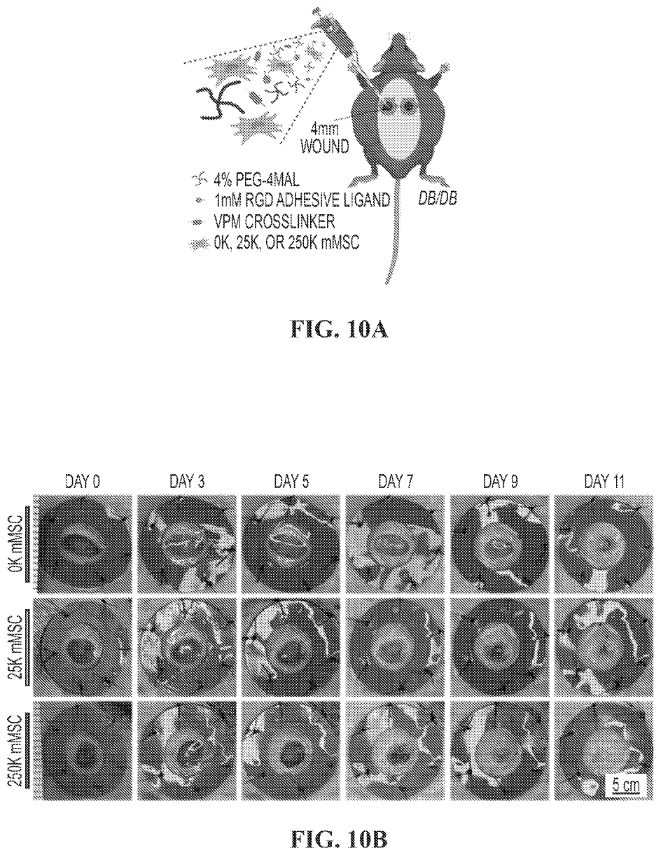

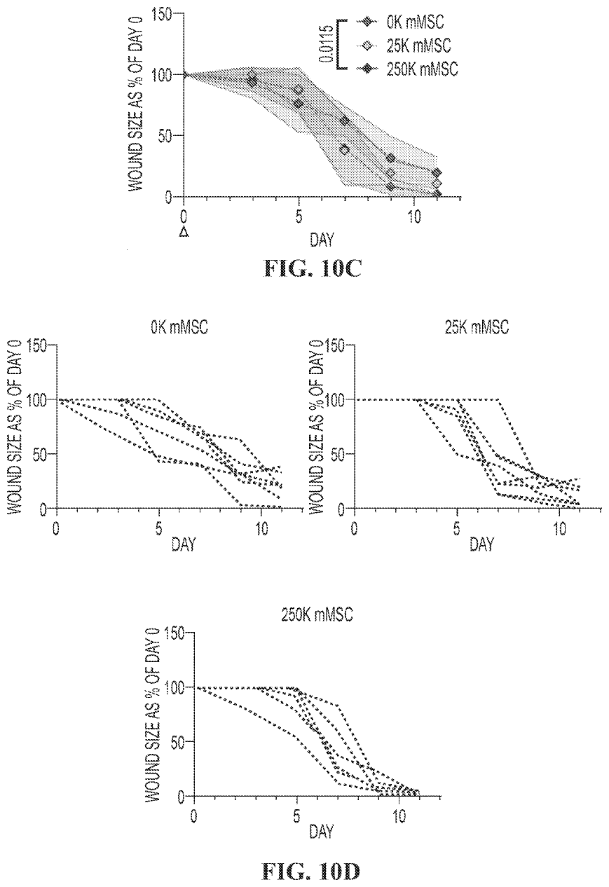

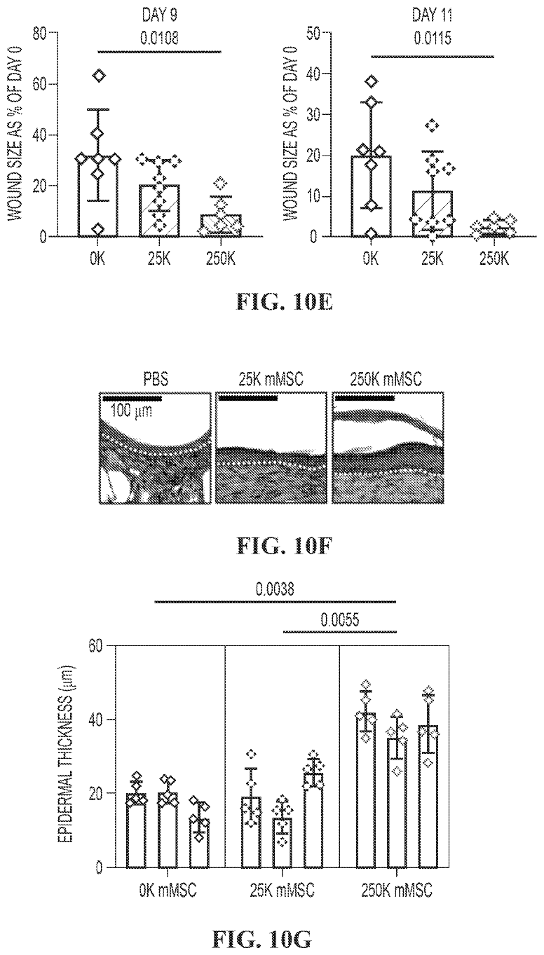

To test whether delivery of mMSC within 75% PEG-4eMAL hydrogels enhances diabetic cutaneous wound healing, 4 mm full-thickness skin wounds were created in the dorsal region of diabetic db/db mice, and two different doses of mMSC, 2.5×10⁴ or 2.5×10⁵ mMSC per 30 μL gel, were delivered in 75% PEG-4eMAL hydrogels (FIG. 10A).

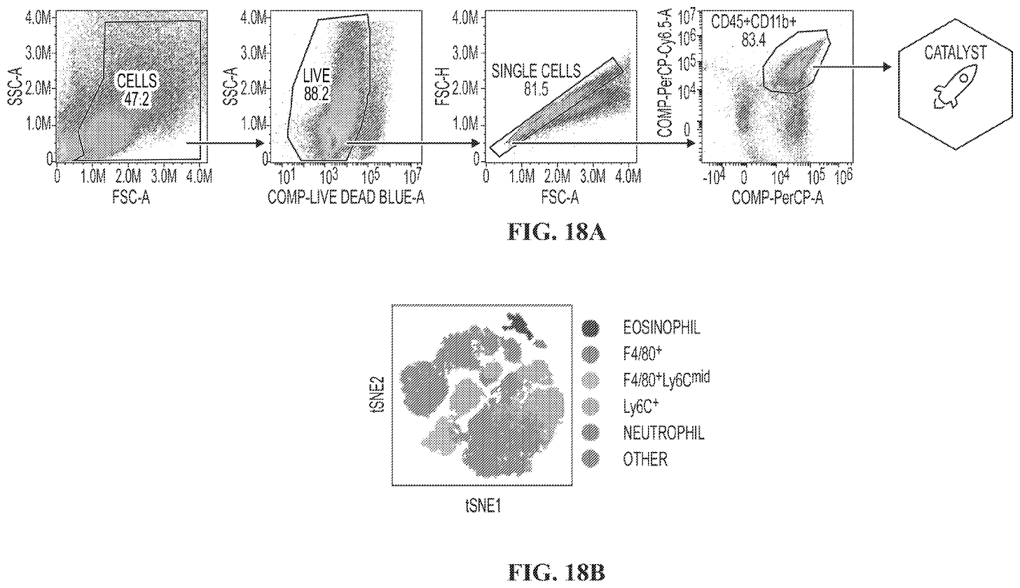

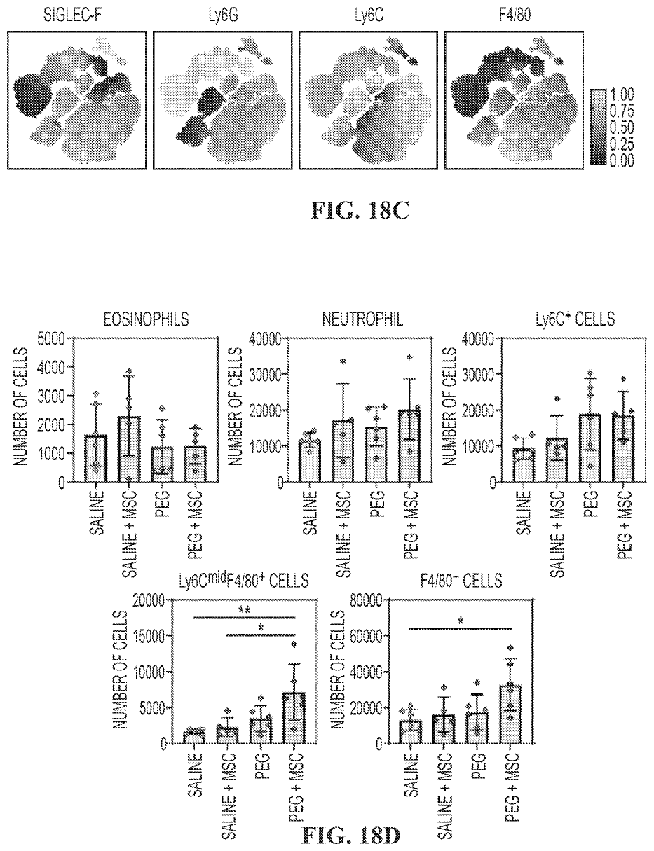

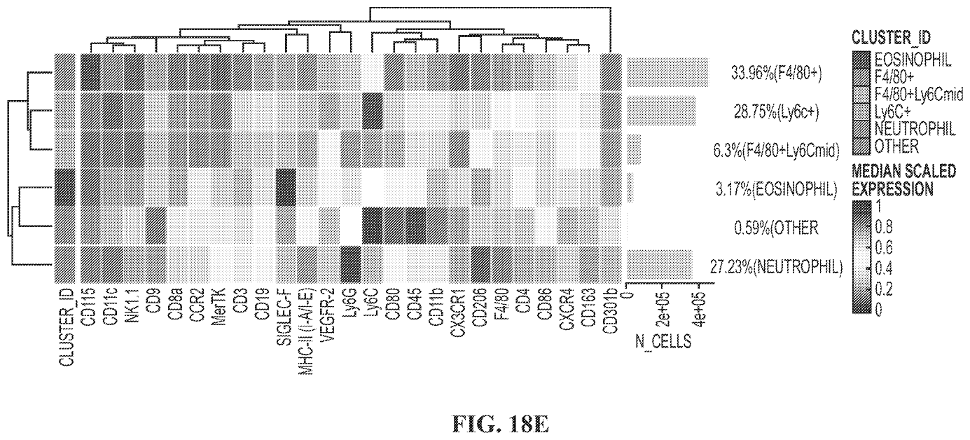

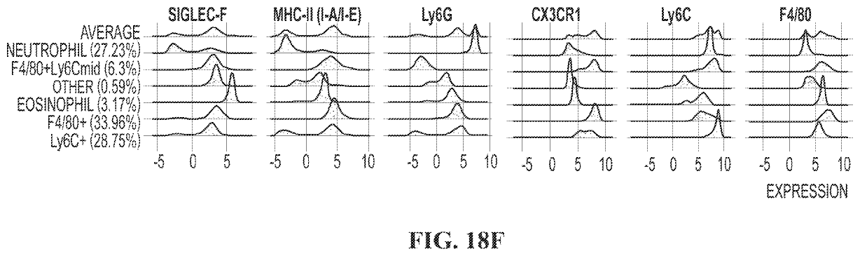

Hydrogels without MSC (Ok mMSC group) were included as controls. Closure of the wounds treated with the higher dose of 2.5×10⁵ cells (250k mMSC group) was significantly higher than the cell-free (Ok mMSC group) on days 9 and 11 (FIGS. 18B-10E). The lower dose of 2.5×10⁴ mMSC (25k mMSC group) showed intermediate levels of wound closure. To evaluate wound healing quality, epidermal thickness was measured from H&E-stained sections of dermal wounds. The average epidermal thicknesses were 18.0±3.9 μm for control hydrogel-only (Ok mMSC) wounds, 19.5±6.1 μm for wounds treated with 25k mMSC in hydrogel, and 38.7±3.5 μm for wounds treated with 250k mMSC in hydrogel (FIGS. 10F-10G). The increased epidermal thickness of wounds treated with 250k mMSC in hydrogels compared to wounds treated with hydrogel-only and 25k mMSC in hydrogel indicates enhanced re-epithelialization and improved healing. These results demonstrate that mMSC delivered in hydrolytic hydrogels enhance cutaneous wound healing in diabetic mice in a dose-dependent manner.

Hydrogel-Delivered mMSC Alter the Immune Response within Cutaneous Wounds

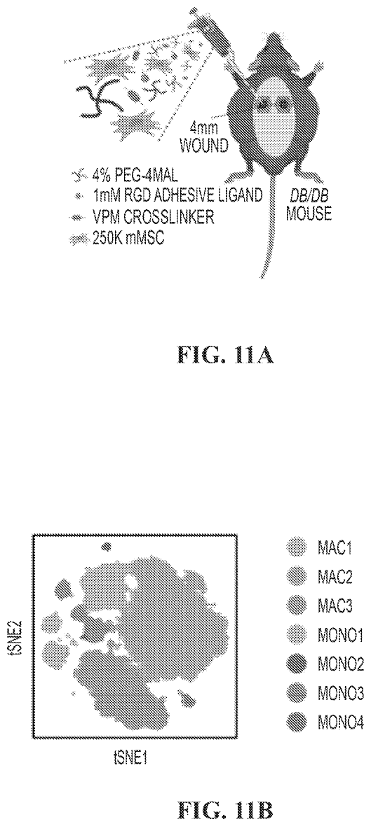

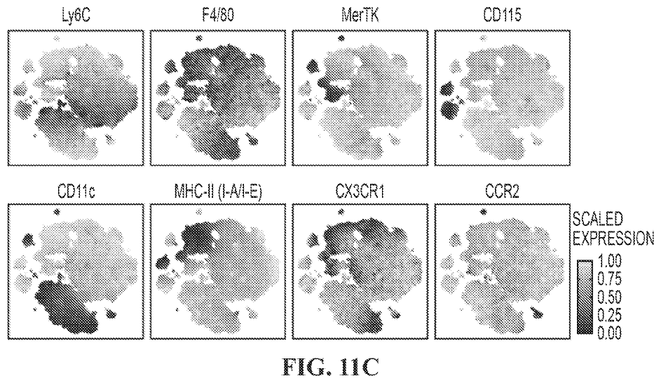

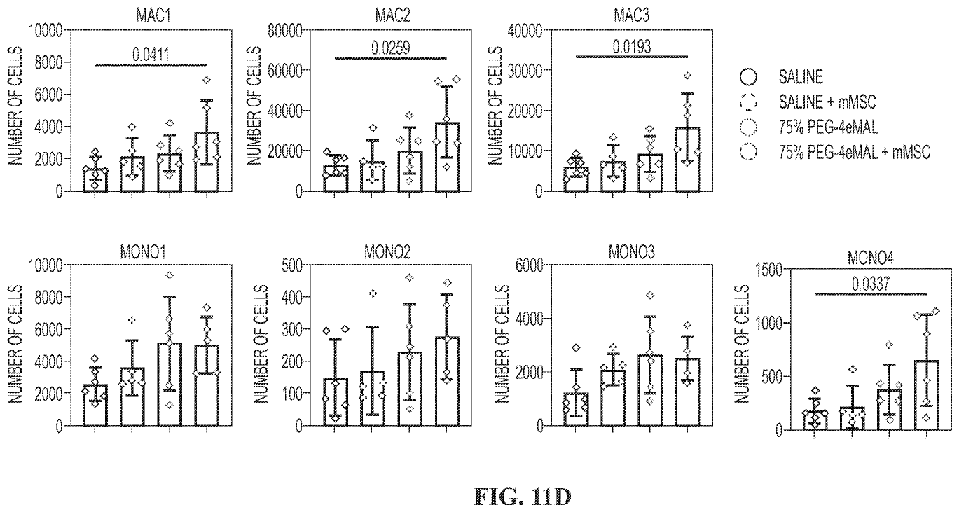

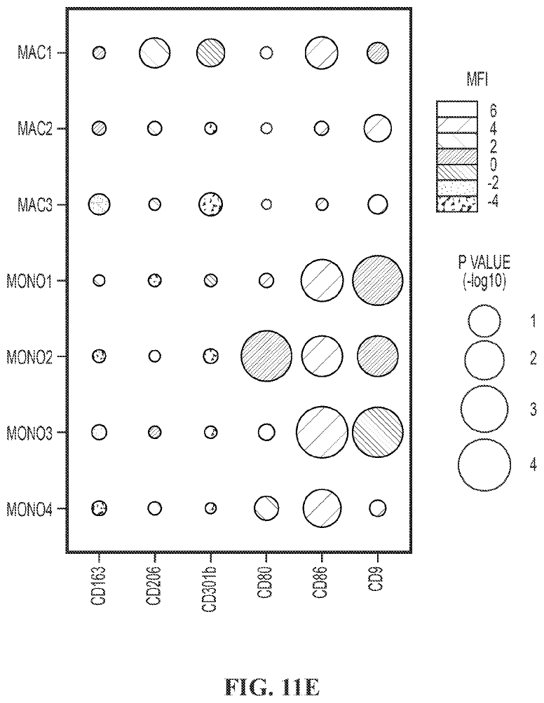

As a productive inflammatory response is a key driver of successful wound repair, we next explored whether hydrolytic hydrogel-delivered mMSC modulate the local wound immune response in diabetic mice. As in the wound healing studies, 4 mm full-thickness skin wounds were created in the dorsal region of diabetic db/db mice. Hydrolytic 75% PEG-4eMAL hydrogels or saline were delivered to the wounds with or without 2.5×10⁵ mMSC (FIG. 11A). On day 3 post-treatment, the wounds and immediately surrounding cutaneous tissue were explanted and infiltrating immune cells were isolated for analysis by spectral flow cytometry. Analysis of the spectral flow data was performed using the CATALYST package in R to call the FlowSOM and tSNE algorithms.

The CD45⁺CD11b⁺ myeloid population was identified using bivariate gating, and FlowSOM was used to cluster the myeloid population into 6 main clusters (FIGS. 18A, 18B, 18E). These clusters, eosinophils, neutrophils, Ly6C⁺ monocytes, Ly6C^{mid}F4/80⁺ double-positive monocytes, F4/80⁺ macrophages, and other CD11b+ cells, were annotated on the basis of expression of 4 population-defining surface markers: Siglec-F, Ly6G, Ly6C, and F4/80 (FIGS. 18C, 18F). There was no difference in the number of cells in the eosinophil, neutrophil, or Ly6C⁺ monocyte clusters among groups (FIG. 18D). Notably, there was a significant increase in the number of Ly6C^{mid}F4/80⁺ double-positive monocytes and F4/80+ macrophages in the hydrolytic gel+mMSC group compared to the cell-free saline control. Additionally, there were significantly more Ly6C^{mid}F4/80⁺ double-positive monocytes infiltrating wounds with hydrogel-delivered mMSC compared to saline-delivered mMSC. There was no difference between the cell-free saline control and the saline-delivered mMSC or cell-free hydrogel conditions, indicating that the combined delivery of mMSC within a hydrolytic hydrogel is necessary for driving these immunological changes.

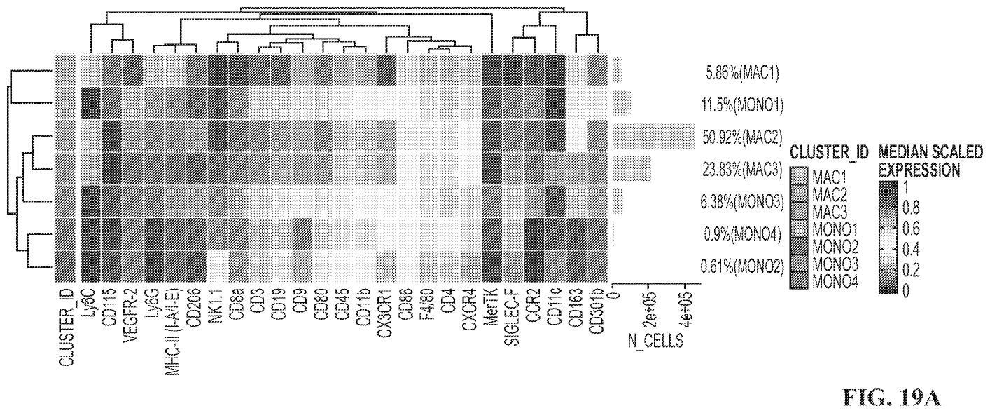

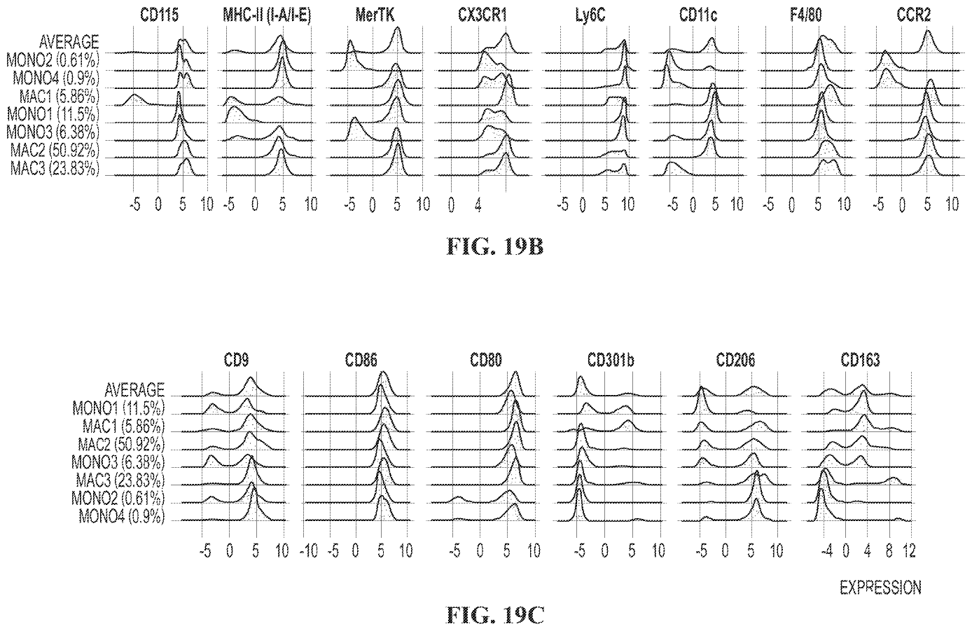

We further characterized the phenotypes of wound-infiltrating mononuclear phagocytes in wounds containing hydrolytic hydrogels with or without mMSC compared to the saline controls. Using the same dataset, the Ly6C⁺ monocyte, Ly6C^{mid}F4/80⁺ double-positive monocyte, and F4/80⁺ macrophage populations were combined and FlowSOM was run to cluster them into subpopulations on the basis of expression of 8 surface markers (Ly6C, F4/80, MerTK, CD115, CD11c, MHC-II, CX3CR1, and CCR2) frequently used to differentiate between different subpopulations and differentiation states of mononuclear phagocytes (FIGS. 11B, 11C, 19A, 19B). This FlowSOM analysis identified 7 main clusters, annotated as either Mono (monocyte, Ly6C^{hi}F4/80⁻CX3CR1^{lo}) or Mac (macrophage, Ly6C F4/80⁺CX3CR1⁺). Similar to the initial analysis of myeloid populations, there was an increase in the number of cells in all Mac clusters and cluster Mono 4 in the hydrolytic gel+mMSC group compared to the cell-free saline control, and no differences between any of the other groups (FIG. 11D).

We then examined the expression of 6 surface markers associated with different macrophage phenotypes within the 7 FlowSOM-identified clusters. These markers included M1 markers CD80 and CD86 and M2 markers CD163 and CD206 [43]. Additionally, the markers CD301 and CD9 were assessed as they have previously been used to differentiate between pro-regenerative and pro-inflammatory macrophages in the context of biomaterial implantation [44]. There were clear differences in the expression patterns of these markers among the different Mono and Mac clusters (FIGS. 11E, 19C). Additionally, the p-values of the difference between the hydrolytic gel+mMSC group compared to the cell-free saline control for CD80, CD86, and CD9 were notably lower in the Mono clusters than the Mac clusters (FIG. 11E). Considering the differences in expression for CD9 and CD86 in more detail, their expression in many Mono clusters is significantly higher in the hydrolytic gel+mMSC group compared to other three groups (FIG. 11F). Interestingly, whereas the monocytes in the hydrolytic gel+mMSC group appear to be expressing more pro-inflammatory/pro-fibrotic markers (CD80, CD86, CD9) than monocytes in the other groups, this is also the group with the best wound healing (FIGS. 9B-9E).

Taken together, these data demonstrate that delivery of mMSC within hydrolytic hydrogels drives immunomodulation at the site of a diabetic, full-thickness skin wound. Furthermore, an enhanced early inflammatory response at the site of the wound is correlated with enhanced wound healing at later time points.

### DISCUSSION

Hydrogel platforms for cell delivery and wound repair applications are limited by slow degradation and poor tissue ingrowth. Whereas hydrogels with macroporous structures have been developed to overcome this issue, these scaffolds still enable little control over the degradation kinetics of the remaining hydrogel [20, 21, 23] which could impair complete tissue repair. In this example, we designed and evaluated a synthetic ester-linked PEG-4MAL hydrogel platform for applications in therapeutic cell delivery and wound repair. We show that by modulating the ratio of ester to amide linkages within the PEG-4MAL macromer, the in vivo degradation rate of these hydrogels can be easily tuned in both healthy and diabetic mice. Whereas slow-degrading amide-linked hydrogels severely impaired cutaneous wound healing, the enhanced degradation rates of ester-linked, hydrolytic gels resulted in wound repair on time scales similar to hydrogel-free controls, thus demonstrating the importance of biomaterial degradation kinetics on successful wound repair.

We demonstrated the limitations of bolus delivery of therapeutic cells, showing that mMSC delivered within hydrolytic gels persisted significantly longer than mMSC delivered in saline, consistent with previous literature [15, 41, 42]. Additionally, we showed that the delivery of mMSC within hydrolytic gel formulations enhanced mMSC persistence compared to mMSC delivery in non-hydrolytic gels where mMSC were rapidly cleared within the first 3 days. As PEG hydrogels degrade, their mesh size increases [36]. Thus, the enhanced mMSC persistence in our hydrolytic gels is consistent with other studies that have shown that denser hydrogels or dense hydrogels lacking macropores result in reduced MSC viability/persistence in vivo [41, 42]. This reduced cellular viability is likely due in part to decreased nutrient transport in hydrogels with denser meshes. Hydrolytic hydrogels with tunable degradation rates are able to overcome this limitation of other synthetic hydrogel systems by degrading relatively rapidly, while still maintaining cellular persistence at the site of delivery significantly beyond what bolus injection is able to achieve.

We hypothesized that tuning hydrolytic gel degradation rates to enable tissue ingrowth and wound healing and enhancing mMSC persistence would promote enhanced diabetic cutaneous wound closure. Indeed, we show that ester-linked hydrogel delivery of mMSC to cutaneous wounds in diabetic mice improves wound repair in this model. Both the time to wound closure and the overall quality of wound healing, as assessed by thickness of the new epithelial layer, were significantly enhanced in hydrolytic hydrogels containing 250,000 mMSC compared to hydrolytic hydrogels alone.

As mMSC are known to be highly immunomodulatory, we hypothesized that the enhanced wound repair seen upon hydrolytic delivery of mMSC to diabetic full-thickness skin wounds is related to a differential immune response. Successful wound healing is dependent on tightly controlled inflammatory responses, with the role of macrophages in this process being well appreciated [43]. Upon injury, circulating monocytes are rapidly recruited from the blood to the site of injury. Once there, they differentiate into pro-inflammatory M1 macrophages that mount an initial inflammatory response, clearing debris, performing antimicrobial functions, and promoting progenitor cell and further immune cell recruitment [45]. This is followed by a transition of macrophages to a pro-regenerative M2 phenotype that drives angiogenesis and tissue remodeling [46]. Immune analysis of diabetic full-thickness skin wounds showed significant differences in the myeloid immune response in wounds with hydrolytic gel-delivered mMSC compared to saline control wounds. In general, wounds with hydrolytic hydrogel-delivered mMSC had significantly increased monocyte and macrophage recruitment compared to saline control wounds. Additionally, the monocytes infiltrating wounds containing hydrolytic gel-delivered mMSC expressed significantly more pro-inflammatory/pro-fibrotic markers (CD80, CD86, CD9) than monocytes in the other groups (saline-delivered mMSC, and cell-free hydrolytic hydrogel). This immune analysis was done at a relatively early time point (3 days post-wounding), so these results highlight the need for an acute inflammatory phase post-injury to recruit immune cells, initiate vascularization, and lay down extracellular matrix scaffolding that ultimately enable successful wound repair [47, 48].

In addition to their tunable degradation properties, ester-linked PEG-4MAL hydrogels have all of the other advantages of the non-hydrolytic amide-linked PEG-4MAL system including cytocompatibility, modularity, and injectability [11, 31, 49]. Additionally, as we have previously shown that we can tether a variety of proteins into PEG-4MAL gels [32, 34, 35, 50], this hydrolytic hydrogel system could provide a way to achieve finer control over in vivo protein delivery rates. Thus, this versatile hydrogel system is amenable to delivery of a variety of cell types and proteins. Poor synthetic hydrogel degradation is one of the key challenges remaining in developing fully-defined, translatable hydrogel platforms. The implementation of hydrolytically degradable ester-linkers as a means of tuning synthetic hydrogel degradation rates represents an important next step in the development of next-generation materials for biomedical applications.

### REFERENCES FROM EXAMPLES

The below references are incorporated herein by reference in their entirety for all purposes:
[1] S. M. Gruss, K. Nhim, E. Gregg, M. Bell, E. Luman, A. Albright, Public Health Approaches to Type 2 Diabetes Prevention: the US National Diabetes Prevention Program and Beyond, Current Diabetes Reports 19 (9) (2019) 78.
[2] L. S. Geiss, Y. Li, I. Hora, A. Albright, D. Rolka, E. W. Gregg, Resurgence of diabetes-related nontraumatic lower-extremity amputation in the young and middle-aged adult US population, Diabetes Care 42 (1) (2019) 50-54.
[3] D. Jiang, Y. Qi, N. G. Walker, A. Sindrilaru, A. Hainzl, M. Wlaschek, S. MacNeil, K. Scharffetter-Kochanek, The effect of adipose tissue derived MSCs delivered by a chemically defined carrier on full-thickness cutaneous wound healing, Biomaterials 34 (10) (2013) 2501-2515.
[4] V. Falanga, S. Iwamoto, M. Chartier, T. Yufit, J. Butmarc, N. Kouttab, D. Shrayer, P. Carson, Autologous bone marrow-derived cultured mesenchymal stem cells delivered in a fibrin spray accelerate healing in murine and human cutaneous wounds, Tissue engineering 13 (6) (2007) 1299-1312.
[5] K. McFarlin, X. Gao, Y. B. Liu, D. S. Dulchavsky, D. Kwon, A. S. Arbab, M. Bansal, Y. Li, M. Chopp, S. A. Dulchavsky, Bone marrow-derived mesenchymal stromal cells accelerate wound healing in the rat, Wound Repair and Regeneration 14 (4) (2006) 471-478.
[6] L. Mazini, L. Rochette, B. Admou, S. Amal, G. Malka, Hopes and limits of adipose-derived stem cells (ADSCs) and mesenchymal stem cells (MSCs) in wound healing, International journal of molecular sciences 21 (4) (2020) 1306.
[7] K. C. Rustad, V. W. Wong, M. Sorkin, J. P. Glotzbach, M. R. Major, J. Rajadas, M. T. Longaker, G. C. Gurtner, Enhancement of mesenchymal stem cell angiogenic capacity and stemness by a biomimetic hydrogel scaffold, Biomaterials 33 (1) (2012) 80-90.
[8] S. Chen, J. Shi, M. Zhang, Y. Chen, X. Wang, L. Zhang, Z. Tian, Y. Yan, Q. Li, W. Zhong, M. Xing, L. Zhang, L. Zhang, Mesenchymal stem cell-laden anti-inflammatory hydrogel enhances diabetic wound healing, Scientific Reports 5 (1) (2015) 18104.
[9] H. Bai, N. Kyu-Cheol, Z. Wang, Y. Cui, H. Liu, H. Liu, Y. Feng, Y. Zhao, Q. Lin, Z. Li, Regulation of inflammatory microenvironment using a self-healing hydrogel loaded with BM-MSCs for advanced wound healing in rat diabetic foot ulcers, Journal of tissue engineering 11 (2020) 2041731420947242.
[10] T. J. Kean, P. Lin, A. I. Caplan, J. E. Dennis, MSCs: Delivery Routes and Engraftment, Cell-Targeting Strategies, and Immune Modulation, Stem Cells Int 2013 (2013) 732742.
[11] A. Y. Clark, K. E. Martin, J. R. García, C. T. Johnson, H. S. Theriault, W. M. Han, D. W. Zhou, E. A. Botchwey, A. J. García, Integrin-specific hydrogels modulate transplanted human bone marrow-derived mesenchymal stem cell survival, engraftment, and reparative activities, Nature Communications 11 (1) (2020) 114.
[12] V. R. Burst, M. Gillis, F. Pütsch, R. Herzog, J. H. Fischer, P. Heid, J. Müller-Ehmsen, K. Schenk, J. W. Fries, C. A. Baldamus, Poor cell survival limits the beneficial impact of mesenchymal stem cell transplantation on acute kidney injury, Nephron Experimental Nephrology 114 (3) (2010) e107-e116.
[13] Y. Iso, J. L. Spees, C. Serrano, B. Bakondi, R. Pochampally, Y.-H. Song, B. E. Sobel, P. Delafontaine, D. J. Prockop, Multipotent human stromal cells improve cardiac function after myocardial infarction in mice without long-term engraftment, Biochemical and biophysical research communications 354 (3) (2007) 700-706.
[14] O. Levy, R. Kuai, E. M. J. Siren, D. Bhere, Y. Milton, N. Nissar, M. De Biasio, M. Heinelt, B. Reeve, R. Abdi, M. Alturki, M. Fallatah, A. Almalik, A. H. Alhasan, K. Shah, J. M. Karp, Shattering barriers toward clinically meaningful MSC therapies, Science Advances 6 (30) (2020) eaba6884.
[15] A. K. Grosskopf, L. Labanieh, D. D. Klysz, G. A. Roth, P. Xu, O. Adebowale, E. C. Gale, C. K. Jons, J. H. Klich, J. Yan, C. L. Maikawa, S. Correa, B. S. Ou, A. I. d′Aquino, J. R. Cochran, O. Chaudhuri, C. L. Mackall, E. A. Appel, Delivery of CAR-T cells in a transient injectable stimulatory hydrogel niche improves treatment of solid tumors, Science Advances 8 (14) (2022) eabn8264.
[16] M. P. Nikolova, M. S. Chavali, Recent advances in biomaterials for 3D scaffolds: A review, Bioact Mater 4 (2019) 271-292.
[17] J. Padmanabhan, T. R. Kyriakides, Nanomaterials, Inflammation, and Tissue Engineering, WIREs Nanomedicine and Nanobiotechnology 7 (3) (2015) 355-370. M. P. Lutolf, J. L. Lauer-Fields, H. G. Schmoekel, A. T. Metters, F. E. Weber, G. B. Fields,
[18] J. A. Hubbell, Synthetic matrix metalloproteinase-sensitive hydrogels for the conduction of tissue regeneration: engineering cell-invasion characteristics, Proceedings of the National Academy of Sciences 100 (9) (2003) 5413-5418.
[19] D. R. Griffin, W. M. Weaver, P. O. Scumpia, D. Di Carlo, T. Segura, Accelerated wound healing by injectable microporous gel scaffolds assembled from annealed building blocks, Nature materials 14 (7) (2015) 737-744.
[20] D. R. Griffin, M. M. Archang, C.-H. Kuan, W. M. Weaver, J. S. Weinstein, A. C. Feng, A. Ruccia, E. Sideris, V. Ragkousis, J. Koh, M. V. Plikus, D. Di Carlo, T. Segura, P. O. Scumpia, Activating an adaptive immune response from a hydrogel scaffold imparts regenerative wound healing, Nature Materials 20 (4) (2021) 560-569.
[21] N. Huebsch, E. Lippens, K. Lee, M. Mehta, Sandeep T. Koshy, Max C. Darnell, R. M. Desai, Christopher M. Madl, M. Xu, X. Zhao, O. Chaudhuri, C. Verbeke, Woo S. Kim, K. Alim, A. Mammoto, Donald E. Ingber, Georg N. Duda, David J. Mooney, Matrix elasticity of void-forming hydrogels controls transplanted-stem-cell-mediated bone formation, Nature Materials 14 (12) (2015) 1269-1277.
[22] S. A. Bencherif, R. W. Sands, D. Bhatta, P. Arany, C. S. Verbeke, D. A. Edwards, D. J. Mooney, Injectable preformed scaffolds with shape-memory properties, Proceedings of the National Academy of Sciences 109 (48) (2012) 19590-19595.
[23] G. Theocharidis, S. Rahmani, S. Lee, Z. Li, A. Lobao, K. Kounas, X.-L. Katopodi, P. Wang, S. Moon, I. S. Vlachos, M. Niewczas, D. Mooney, A. Veves, Murine macrophages or their secretome delivered in alginate dressings enhance impaired wound healing in diabetic mice, Biomaterials (2022) 121692.
[24] M. D. Hoffman, A. H. Van Hove, D. S. Benoit, Degradable hydrogels for spatiotemporal control of mesenchymal stem cells localized at decellularized bone allografts, Acta Biomater 10 (8) (2014) 3431-41.
[25] Y. Wang, S. Zhang, D. S. W. Benoit, Degradable poly(ethylene glycol) (PEG)-based hydrogels for spatiotemporal control of siRNA/nanoparticle delivery, J Control Release 287 (2018) 58-66.
[26] C. E. Miksch, N. P. Skillin, B. E. Kirkpatrick, G. K. Hach, V. V. Rao, T. J. White, K. S. Anseth, 4D Printing of Extrudable and Degradable Poly (Ethylene Glycol) Microgel Scaffolds for Multidimensional Cell Culture, Small n/a (n/a) 2200951.
[27] Y. S. Jo, J. Gantz, J. A. Hubbell, M. P. Lutolf, Tailoring hydrogel degradation and drug release via neighboring amino acid controlled ester hydrolysis, Soft Matter 5 (2) (2009) 440-446.
[28] A. E. Rydholm, K. S. Anseth, C. N. Bowman, Effects of neighboring sulfides and pH on ester hydrolysis in thiol-acrylate photopolymers, Acta biomaterialia 3 (4) (2007) 449-455.
[29] S. P. Zustiak, J. B. Leach, Hydrolytically degradable poly(ethylene glycol) hydrogel scaffolds with tunable degradation and mechanical properties, Biomacromolecules 11 (5) (2010) 1348-1357.
[30] M. D. Hunckler, J. D. Medina, M. M. Coronel, J. D. Weaver, C. L. Stabler, A. J. García, Linkage Groups within Thiol-Ene Photoclickable PEG Hydrogels Control In Vivo Stability, Advanced Healthcare Materials 8 (14) (2019) 1900371.
[31] E. A. Phelps, N. O. Enemchukwu, V. F. Fiore, J. C. Sy, N. Murthy, T. A. Sulchek, T. H. Barker, A. J. García, Maleimide cross-linked bioactive peg hydrogel exhibits improved reaction kinetics and cross-linking for cell encapsulation and in situ delivery, Advanced materials 24 (1) (2012) 64-70.
[32] J. R. García, M. Quirós, W. M. Han, M. N. O'Leary, G. N. Cox, A. Nusrat, A. J. García, IFN-γ-tethered hydrogels enhance mesenchymal stem cell-based immunomodulation and promote tissue repair, Biomaterials 220 (2019) 119403.
[33] E. A. Phelps, D. M. Headen, W. R. Taylor, P. M. Thulé, A. J. García, Vasculogenic bio-synthetic hydrogel for enhancement of pancreatic islet engraftment and function in type 1 diabetes, Biomaterials 34 (19) (2013) 4602-4611.
[34] J. R. García, A. Y. Clark, A. J. García, Integrin-specific hydrogels functionalized with VEGF for vascularization and bone regeneration of critical-size bone defects, Journal of Biomedical Materials Research Part A 104 (4) (2016) 889-900.
[35] J. D. Medina, G. F. Barber, M. M. Coronel, M. D. Hunckler, S. W. Linderman, M. J. Quizon, V. Ulker, E. S. Yolcu, H. Shirwan, A. J. García, A hydrogel platform for co-delivery of immunomodulatory proteins for pancreatic islet allografts, J Biomed Mater Res A 110 (11) (2022) 1728-1737.
[36] W. M. Han, S. E. Anderson, M. Mohiuddin, D. Barros, S. A. Nakhai, E. Shin, I. F. Amaral, A. P. Pêgo, A. J. García, Y. C. Jang, Synthetic matrix enhances transplanted satellite cell engraftment in dystrophic and aged skeletal muscle with comorbid trauma, Science Advances 4 (8) (2018) eaar4008.
[37] K. E. Martin, P. P. Kalelkar, M. M. Coronel, H. S. Theriault, R. S. Schneider, A. J. García, Host type 2 immune response to xenogeneic serum components impairs biomaterial-directed osteo-regenerative therapies, Biomaterials (2022) 121601.
[38] L. Dunn, H. C. Prosser, J. T. Tan, L. Z. Vanags, M. K. Ng, C. A. Bursill, Murine model of wound healing, JoVE (Journal of Visualized Experiments) (75) (2013) e50265.
[39] J. Yan, G. Tie, S. Wang, A. Tutto, N. DeMarco, L. Khair, T. G. Fazzio, L. M. Messina, Diabetes impairs wound healing by Dnmt1-dependent dysregulation of hematopoietic stem cells differentiation towards macrophages, Nature communications 9 (1) (2018) 1-13.
[40] J. Michaels, S. S. Churgin, K. M. Blechman, M. R. Greives, S. Aarabi, R. D. Galiano, G. C. Gurtner, db/db mice exhibit severe wound-healing impairments compared with other murine diabetic strains in a silicone-splinted excisional wound model, Wound repair and regeneration 15 (5) (2007) 665-670.
[41] A. K. Grosskopf, G. A. Roth, A. A. A. Smith, E. C. Gale, H. L. Hernandez, E. A. Appel, Injectable supramolecular polymer-nanoparticle hydrogels enhance human mesenchymal stem cell delivery, Bioengineering & Translational Medicine 5 (1) (2020) e10147.
[42] J. Koh, D. R. Griffin, M. M. Archang, A.-C. Feng, T. Horn, M. Margolis, D. Zalazar, T. Segura, P. O. Scumpia, D. Di Carlo, Enhanced In Vivo Delivery of Stem Cells using Microporous Annealed Particle Scaffolds, Small 15 (39) (2019) 1903147.
[43] K. E. Martin, A. J. García, Macrophage phenotypes in tissue repair and the foreign body response: Implications for biomaterial-based regenerative medicine strategies, Acta Biomaterialia 133 (2021) 4-16.
[44] S. D. Sommerfeld, C. Cherry, R. M. Schwab, L. Chung, D. R. Maestas, Jr., P. Laffont, J. E. Stein, A. Tam, S. Ganguly, F. Housseau, J. M. Taube, D. M. Pardoll, P. Cahan, J. H. Elisseeff, Interleukin-36γ-producing macrophages drive IL-17-mediated fibrosis, Sci Immunol 4 (40) (2019).
[45] M. E. Ogle, C. E. Segar, S. Sridhar, E. A. Botchwey, Monocytes and macrophages in tissue repair: Implications for immunoregenerative biomaterial design, Experimental Biology and Medicine 241 (10) (2016) 1084-1097.
[46] K. L. Spiller, T. J. Koh, Macrophage-based therapeutic strategies in regenerative medicine, Advanced Drug Delivery Reviews 122 (2017) 74-83.
[47] P. L. Graney, S. Ben-Shaul, S. Landau, A. Bajpai, B. Singh, J. Eager, A. Cohen, S. Levenberg, K. L. Spiller, Macrophages of diverse phenotypes drive vascularization of engineered tissues, Science Advances 6 (18) (2020) eaay6391.
[48] C. E. Witherel, D. Abebayehu, T. H. Barker, K. L. Spiller, Macrophage and Fibroblast Interactions in Biomaterial-Mediated Fibrosis, Adv Healthc Mater 8 (4) (2019) e1801451.
[49] R. Cruz-Acuña, M. Quirós, A. E. Farkas, P. H. Dedhia, S. Huang, D. Siuda, V. García-Hernández, A. J. Miller, J. R. Spence, A. Nusrat, Synthetic hydrogels for human intestinal organoid generation and colonic wound repair, Nature cell biology 19 (11) (2017) 1326-1335.
[50] N. M. Beskid, E. M. Kolawole, M. M. Coronel, B. Nguyen, B. Evavold, A. J. García, J. E. Babensee, IL-10-Functionalized Hydrogels Support Immunosuppressive Dendritic Cell Phenotype and Function, ACS Biomaterials Science & Engineering 8 (10) (2022) 4341-4353.
[51] R. S. Schneider, A. C. Vela, E. K. Williams, K. E. Martin, W. A. Lam, A. J. García, High-Throughput On-Chip Human Mesenchymal Stromal Cell Potency Prediction, Advanced Healthcare Materials 11 (2) (2022) 2101995.
[51] C. M. Caroti, H. Ahn, H. F. Salazar, G. Joseph, S. B. Sankar, N. J. Willett, L. B. Wood, W. R. Taylor, A. N. Lyle, A Novel Technique for Accelerated Culture of Murine Mesenchymal Stem Cells that Allows for Sustained Multipotency, Scientific Reports 7 (1) (2017) 13334.
[52] S. Nandi, E. P. Sproul, K. Nellenbach, M. Erb, L. Gaffney, D. O. Freytes, A. C. Brown, Platelet-like particles dynamically stiffen fibrin matrices and improve wound healing outcomes, Biomaterials science 7 (2) (2019) 669-682.
[53] M. Nowicka, C. Krieg, H. Crowell, L. Weber, F. Hartmann, S. Guglietta, B. Becher, M. Levesque, M. Robinson, CyTOF workflow: differential discovery in high-throughput high-dimensional cytometry datasets [version 4; peer review: 2 approved], F1000Research 6 (748) (2019).
[54] S. B. Shah, C. R. Carlson, K. Lai, Z. Zhong, G. Marsico, K. M. Lee, N. E. Félix Vélez, E. B. Abeles, M. Allam, T. Hu, L. D. Walter, K. E. Martin, K. Gandhi, S. D. Butler, R. Puri, A. L. McCleary-Wheeler, W. Tam, O. Elemento, K. Takata, C. Steidl, D. W. Scott, L. Fontan, H. Ueno, B. D. Cosgrove, G. Inghirami, A. J. García, A. F. Coskun, J. L. Koff, A. Melnick, A. Singh, Combinatorial treatment rescues tumour-microenvironment-mediated attenuation of MALTI inhibitors in B-cell lymphomas, Nat Mater 22 (4) (2023) 511-523.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A hydrogel comprising:
a first polymer comprising at least one ester-containing moiety and at least one first crosslinking moiety;
a second polymer comprising at least one amide-containing moiety and at least one second crosslinking moiety; and
and a crosslinker covalently bound to the first polymer and the second polymer via the first crosslinking moiety and the second crosslinking moiety.

2. The hydrogel of claim 1, wherein the first polymer is hydrolytically degradable.

3. (canceled)

4. The hydrogel of claim 1, wherein the first crosslinking moiety and/or the second crosslinking moiety is derived from a maleimide moiety.

5. The hydrogel of claim 1, wherein the first polymer and/or the second polymer comprises a poly(ethylene glycol) backbone.

6. The hydrogel of claim 1, wherein the first polymer and/or the second polymer is a multi-arm polymer.

7. (canceled)

8. The hydrogel of claim 6, wherein the first polymer and/or the second polymer comprises a core covalently bound to each arm.

9. The hydrogel of claim 8, wherein the core is derived from glycerol, pentaerythritol, dipentaerythritol, or tripentaerythritol.

10. The hydrogel of claim 1, wherein at each occurrence the ester-containing moiety comprises a monoester.

11-12. (canceled)

13. The hydrogel of claim 1, wherein the first polymer comprises a compound of Formula I: 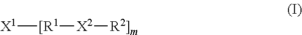
wherein:
X¹ is a core;
R¹ is a poly(ethylene glycol) backbone;
X² is the ester-containing moiety;
R² is the first crosslinking moiety; and
m is an integer selected from 3 to 8.

14-15. (canceled)

16. The hydrogel of claim 1, wherein the second polymer is hydrolytically stable.

17-23. (canceled)

24. The hydrogel of claim 1, wherein the second polymer comprises a compound of Formula II: 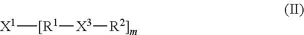
wherein:
X¹ is a core;
R¹ is a poly(ethylene glycol) backbone;
X³ is the amide-containing moiety;
R² is the first crosslinking moiety; and
m is an integer selected from 3 to 8.

25-29. (canceled)

30. The hydrogel of claim 1, wherein the crosslinker is covalently bound to the first crosslinking moiety by a thioether linkage, and/or
wherein the crosslinker is covalently bound to the second crosslinking moiety by a thioether linkage.

31. (canceled)

32. The hydrogel of claim 1, wherein the crosslinker is hydrolytically degradable or hydrolytically stable.

33. The hydrogel of claim 32, wherein the crosslinker is selected from: ethylene glycol bis(mercaptoacetate) (EGBMA); glycol di(3-mercaptopropionate); ethylene bis(thioglycolate); glyceryl dithioglycolate (GDT); and poly(ethylene glycol)-diester dithiol.

34. (canceled)

35. The hydrogel of claim 32, wherein the crosslinker is selected from: 1,4-dithiothreitol (DTT); poly(ethylene glycol)-dithiol (PEG-DT); and 2,2′-(ethyleneoxy) diethanethiol (EDDT).

36. The hydrogel of claim 1, wherein the crosslinker comprises a peptide selected from: GCRDVPMSMRGGDRCG (SEQ ID NO. 1); GCRDGDQGIAGFDRCG (SEQ ID NO. 2); GCRDGPQGIAGODRCG (SEQ ID NO. 3); GCRDGPQGIWGODRCG (SEQ ID NO. 4); GCRDIPESLRAGDRCG (SEQ ID NO. 5); and CVPLSLYSGC (SEQ ID NO. 6).

37-40. (canceled)

41. A therapeutic delivery composition comprising a hydrogel of claim 1 and one or more therapeutic agents.

42-44. (canceled)

45. A method of delivering one or more therapeutic agents to a target site in a subject, the method comprising administering a therapeutically effective amount of a therapeutic delivery composition of claim 41.

46-47. (canceled)

48. A method of promoting tissue growth in a target site in a subject in need thereof, comprising administering to the target site a therapeutically effective amount of a hydrogel of claim 1.

49. (canceled)

50. A method of transplanting at least one cell to a target site in a subject in need thereof, the method comprising administering to the target site an effective amount of a composition comprising the hydrogel claim 1 and the at least one cell.

51. (canceled)


SEQUENCE LISTING (20 sequences)

SEQ ID NO: 1 - protein, 16 aa, synthetic construct
GCRDVPMSMRGGDRCG

SEQ ID NO: 2 - protein, 16 aa, synthetic construct
GCRDGDQGIAGFDRCG

SEQ ID NO: 3 - protein, 16 aa, synthetic construct
GCRDGPQGIAGQDRCG

SEQ ID NO: 4 - protein, 16 aa, synthetic construct
GCRDGPQGIWGQDRCG

SEQ ID NO: 5 - protein, 16 aa, synthetic construct
GCRDIPESLRAGDRCG

SEQ ID NO: 6 - protein, 10 aa, synthetic construct
CVPLSLYSGC

SEQ ID NO: 7 - protein, 7 aa, synthetic construct
GRGDSPC

SEQ ID NO: 8 - protein, 7 aa, synthetic construct
GRDGSPC

SEQ ID NO: 9 - protein, 15 aa, synthetic construct
CGGRKRLQVQLSIRT

SEQ ID NO: 10 - protein, 14 aa, synthetic construct
CGGEGYGEGYIGSR

SEQ ID NO: 11 - protein, 15 aa, synthetic construct
CGGAASIKVAVSADR

SEQ ID NO: 12 - protein, 15 aa, synthetic construct
CGGTWSQKALHHRVP

SEQ ID NO: 13 - protein, 15 aa, synthetic construct
CGGAGQWHRVSVRWG

SEQ ID NO: 14 - protein, 14 aa, synthetic construct
CGKKQRFRHRNRKG

SEQ ID NO: 15 - protein, 14 aa, synthetic construct
CGTLFLAHGRLVFM

SEQ ID NO: 16 - protein, 12 aa, synthetic construct
CGFHVAYVLIKF

SEQ ID NO: 17 - protein, 15 aa, synthetic construct
CGRLVSYNGIIFFLK

SEQ ID NO: 18 - protein, 22 aa, synthetic construct
CGLRRFSTAPFAFIDINDVINF

SEQ ID NO: 19 - protein, 45 aa, synthetic construct
  SITE 25: note = 4Hyp
GYGGGPPGPPGPPGPPGPPGPPGFXGERPPGPPGPPGPPGPPGPC

SEQ ID NO: 20 - protein, 45 aa, synthetic construct
  SITE 25: note = 4Hyp
GYGGGPPGPPGPPGPPGPPGPPGAXGERPPGPPGPPGPPGPPGPC